(19) United States
(12) Patent Application Publication
(10) Pub. No.: US 2012/0259252 A1
(43) Pub. Date: Oct. 11, 2012

(54) Coupling Emulsions for Use With Ultrasound Devices

(75) Inventors: Daniel Thorn-Leeson, New York, NY (US); Bing C. Mei, Mahwah, NJ (US); Mark S. Garrison, Suffern, NY (US); Lisa Lamberty, Hawthorne, NJ (US); Clifford A. Milow, Massapequa, NY (US)

(73) Assignee: AVON PRODUCTS, INC., New York, NY (US)

(21) Appl. No.: 13/514,794

(22) PCT Filed: Oct. 15, 2010
(86) PCT No.: PCT/US10/52803

§ 371 Date: Jun. 8, 2012

Related U.S. Application Data
(60) Provisional application No. 61/289,194, filed on Dec. 22, 2009.

(51) Int. Cl. A61N 7/02; A61F 7/08; A61Q 19/00; A61P 17/00; A61K 8/92; A61K 47/44

(52) U.S. Cl. 601/3; 514/772; 514/772.6; 607/96

(57) ABSTRACT

Methods and cosmetic coupling compositions are provided that can be effectively used in conjunction with an ultrasound or similar device. The compositions and methods allow extended manipulation on the skin and heating without breaking down, pilling or balling and while maintaining a pleasant cosmetic feel and leaving skin feeling soft.

### CROSS REFERENCE TO RELATED APPLICATIONS

This application claims priority to International Application Serial No. PCT/US10/52803 filed Oct. 15, 2010, which claims priority U.S. Provisional Patent Application Ser. No. 61/289,194 filed Dec. 22, 2009, the contents of which are hereby incorporated by reference in their entirety.

### FIELD OF INVENTION

The present invention relates to cosmetic compositions that can be effectively used in conjunction with an ultrasound or similar device to provide a benefit to the skin. The compositions and methods allow extended manipulation on the skin without breaking down, pilling or balling and while maintaining a pleasant aesthetic feel.

### BACKGROUND OF INVENTION

The gradual development of facial wrinkles, whether fine surface lines or deeper creases and folds, is the classic early sign of accumulated skin damage and aging. Premature aging and wrinkling of the skin may be accelerated by excessive exposure to the sun and other elements, overactive facial expression muscles, the frequent use of tobacco products, poor nutrition, or skin disorders. Fine surface wrinkles that progress to deeper creases, deepening facial expression due to repeated skin folding, and deep folds which develop with one's maturity are obvious changes which may combine to portray a less desirable appearance.

The deterioration of human skin due to natural or ‘intrinsic’ ageing is characterized by a number of symptoms including loss of elasticity and reduced metabolic activity. Specifically, the stratum corneum remains unchanged, but the epidermis thins overall, with a flattening of the dermal-epidermal junction resulting in increased fragility of the skin. Dermal thickness and dermal vascularity are decreased; this is accompanied by a decrease in the number and the biosynthetic activity of dermal fibroblasts. Increasing age also has the effect of reducing the response of keratinocytes and fibroblasts to growth factors.

Inflammation is an underlying contributor to the signs of aging skin inflammation (including acne, eczema, contact allergies) can cause the breakdown of collagen, create pigmentary irregularities spiotchiness), and cause scarring. Chronic inflammation appears strongly linked to many preventable and treatable skin diseases and conditions such as visible skin aging. The skin is also subjected to environmental ageing processes. For example, factors such as diet, pollution and smoking are known to affect the rate of skin ageing. However one factor stands out as the most potent “gerontogen”: sunlight. Typical symptoms of photoaging include coarseness, wrinkling, irregular pigmentation, telangiectasia, scaliness and a variety of benign, pre-malignant and malignant neoplasms. Elastosis, recognized as the pebbly goose flesh seen on the neck and upper chest, is due to nodular aggregations of altered elastin fibers in the dermis. A proliferation of increasingly thickened and tangled elastin fibers has been observed in the papillary and reticular dermis of sun-exposed skin.

Ultrasound, and in particular low-intensity ultrasound, can stimulate certain biochemical processes such as the production of new collagen. The production of new collagen is a biochemical endpoint that anti-aging treatments share with the process of wound healing (Davidson et al. (1985) J. Cell Biol. 100:1219-1227.). It has been suggested that mild stimulation at an intensity level below any threshold for injury, with either light (Lee et al. (2007, J. Photochem. Photobiol. 88:51-67.), ultrasound (Zhou et al. (2004) J. Biol. Chem. 52:54463-54469), or heat (Mayes and Holyoak (2008) Rejuvenation Res. 11:461-465) can trigger the cascade of biochemical processes necessary for the restructuring and production of new collagen. These processes are similar to what happens during the process of wound healing, but these biochemical events are triggered without actually causing trauma. Cell surface receptors called integrins, can be found on the surface of fibroblasts that produce collagen in the dermis (Fisher, et al. (2008) Arch. Dermatol. 144:666-672; Ingber (2003) Proc. Natl. Acad. Sci, USA 100:1472-1474). Integrins can mediate cell-generated forces or external stresses by forming so-called focal adhesions between the extracellular matrix outside the cell and the cytoskeleton inside the cell. Specifically, the formation of focal adhesions can activate the intracellular signal transduction pathways that regulate fibroblast metabolism, including the production of new collagen.

Low intensity ultrasound has been observed to promote bone and wound healing by causing fibroblasts to proliferate and produce more collagen (Rubin, et al. (2001) J. Bone Joint Surg. Am. 83A:259-270; Demir, et al. (200.4) J. Rehabil, Res. Dev. 41:721-728). This hypothesis was tested by Zhou et al. who demonstrated that exposure of human skin fibroblasts in vitro to low intensity ultrasound (1.5 MHz, 30 mW/cm², 1 kHz pulse rate, 6-11 minute exposure) increases cell proliferation (Thou et al. (2004) J. Biol. Chem. 52:54463-54469). Additionally, exposure of hairless mice in vivo with ultrasound (1 MHz, 30 mW/cm², CW, 10-minute single exposure) was shown to lead to stimulate epidermal expression of cytokines IL-1α, TNF-α, and TGF-β (Choi et al. (2003) J. Invest. Dermatol. 121:1138-1144), that have been suggested to play a key role in collagen re-structuring and synthesis. Finally, it has been reported that a repetitive mild heat shock regime (41° C., 60 minutes, 4 repeats) increases dermal fibroblast activity and collagen production in vitro (Mayes and Holyoak (2008) Rejuvenation Res. 11:461-465), suggesting that the modest thermal effects of low intensity ultrasound may also play a role in its observed clinical benefits.

The depth of penetration into skin of ultrasound is inversely related to the frequency. Ultrasound at lower frequency is believed to provide effect towards deeply into the tissue, thus is effectively used for diagnosis, while higher frequency is believed to provide more effect towards the surface of the skin. PCT publication WO 88/00001 describes the use of ultrasound at a frequency of no more than about 2.5 MHz for effectively delivering drugs to the circulatory system.

The use of ultrasound to deliver agents transcutaneously, generally termed “sonophoresis” or “phonophoresis,” is known in the art, for example in GB publication 1577551, PCT publication WO 88/00001, PCT publication WO 91/12772, PCT publication WO 94/08655, U.S. Pat. No. 5,267,985, PCT publication WO 97/04832, U.S. Pat. No. 5,445,611, PCT publication WO 97/40679, PCT publication WO 99/51295, U.S. Pat. No. 6,066,123, Japanese patent publication A-11-335271, U.S. publication 2002-55702, and PCT publication WO 00/21605. Ultrasound devices for use by individual users are also known. For example, PCT application WO 98/51255 teaches an ultrasound application device which has multiple safety features suitable for use by a layperson without the aid of a specialist.

U.S. Pat. No. 7,001,355 to the Procter & Gamble Company describes an ultrasound device and composition that is not an emulsion comprising: (a) a skin active agent; (b) a viscosifying agent that provides the composition a viscosity of from about 1,000 mPas to about 1,000,000 mPas; (c) from about 0.1% to about 30% of a water-soluble humectant; and (d) an aqueous carrier; wherein the composition is substantially free of surfactants.

U.S. Publication No. 20080051680 describes an ultrasound delivery apparatus comprising flexible arrays of transducers and methods and topical compositions for the treatment of skin, in particular for the treatment of cosmetic skin conditions and to improve the appearance of sun damaged and/or aged skin using one or more anti-glycation agent, one or more anti-oxidant, a dermatologically acceptable excipient and optionally one or more substances capable of inducing expression of a molecular chaperone.

U.S. Publication 20080294073 describes a system for non-ablative acne treatment and prevention utilizing ultrasound energy which is targeted at a region of interest to treat existing acne and prevent future acne from forming by reducing sebum, increasing perfusion at the region of interest, denaturing proteins at the region of interest, creating an uninhabitable environment at the region of interest, initiating programmed cell death at the region of interest and the initiation of mechanical effects at the region of interest.

U.S. Pat. No. 6,113,559 discloses a method and apparatus of reducing wrinkles by application of a focused ultrasound beam to a region of skin to heat the tissue in order to stimulate or irritate the dermis layer. EP 0 695 559, relates to multifunctional equipment for treatments of cellulite. GB 2303552 discloses ultrasound apparatus useful for the non-invasive reduction of cellulite. U.S. Pat. No. 5,507,790 discloses apparatus for focusing ultrasound energy such that the temperature of a site within the patient's subcutaneous adipose tissue layer is raised.

Ultrasound can be used to improve transdermal drug delivery. WO 99/34857 and U.S. Pat. No. 4,767,402 disclose transdermal drug delivery of various active agents using specific power density.

In order for ultrasound waves to be effectively delivered to the tissue, a so-called “coupling gel” is required for which the acoustic impedance is similar to that of the tissue. The coupling gel or “ultrasound gel” is typically a water or glycerin based get that is applied onto the skin at the body site to be diagnosed or treated. The ultrasonic probe is subsequently applied to the skin.

However, the aesthetic properties of the ultrasound gel are unfavorable, which presents a major obstacle for the use of ultrasound for home-use cosmetic applications, where the user, at a minimum, demands an in-use experience that is aesthetically neutral, and preferably pleasant. Specifically, gets tend to “sit” on the skin without being absorbed while feeling “sticky” and “wet.” Mineral oil has been used as an acoustic coupling medium for clinical magnetic resonance guided focused ultrasound however the oil significantly reduced the strength of acoustic waves and increased the temperature of the skin when compared with traditional gel-based coupling media (Gomy, et al. (2007) Phys. Med. Biol. 52:N13-N19)

Although cosmetic formulations are designed to have pleasant in-use tactile properties on the skin, i.e. provide a pleasant “skin feel,” their use as a coupling medium/fluid for a hand-held ultrasound device presents a number of problems. A cosmetic ultrasound treatment requires moving the ultrasound probe across the skin, for instance by moving the probe over the surface in circular or linear motions, for periods of several minutes, much longer than the typical application of a topical cosmetic product. The extended “rubbing” of the formulation between the probe and the skin results in “pilling” or “balling,” which is a phase separation of the formulation resulting in the formation of small solid deposits on the skin resulting from the mechanical friction and by evaporation of solvent components enhanced by the rubbing action. Such balling or pilling is highly undesirable from an in-use aesthetics perspective.

The pilling effect of formulations can be exacerbated by heating of the ultrasound probe. The piezoelectric element that generates the ultrasound waves tends to generate heat, which can result in heating of the ultrasonic probe, which is in direct contact with the piezoelectric element. Any heating of the probe will be transferred to the applied formula and may exacerbate pilling/balling.

U.S. Pat. No. 7,022,316 provides certain topically applicable, non-pilling UV-photoprotecting sunscreen compositions for UV-photoprotecting human skin and/or hair that contain an effective amount of at least one UV-A and/or UV-B screening agent and an effective non-pining amount of an acrylates/C₁₂₋₂₂ alkylmethacrylate copolymer. Typically described are compositions that include a tetrapolymer of methacrylic acid, methyl methacrylate, butyl acrylate and cetyl-eicosinyl methacrylate, formulated into a topically applicable, cosmetically/dermatologically acceptable vehicle, diluent or carrier.

U.S. Pat. No. 5,115,805 to Cygnus describes certain methods of enhancing drug delivery via ultrasound including in conjunction with chemical permeation enhancers and/or with iontophoresis

Based on the foregoing, there is a need for a skin care composition that can be safely and effectively used in conjunction with an ultrasound or similar device to provide safe and effective skin care treatment benefit. Specifically, there is a need for a composition which, when used in combination with an ultrasound applying apparatus, can effectively deliver the ultrasound to the skin while providing smoothness and moisture to the skin without leaving the skin feel sticky and without pilling upon prolonged exposure to heat and friction.

It is therefore an object of the present invention to provide improved compositions and methods of use to improve the appearance of skin, including by reducing the appearance of fine lines and wrinkles.

### SUMMARY OF INVENTION

In accordance with the foregoing objectives and others, the present invention provides systems and compositions that allow the use of ultrasound or similar techniques for cosmetic applications while avoiding the unpleasant feel of water-based gels and while avoiding the pilling or balling that occurs when a typical cosmetic emulsion is subjected to extended heat or friction. It has surprisingly been found that emulsions with certain rheological characteristics are resistant to pilling or balling and are therefore particularly useful in conjunction with elements that cause friction during cosmetic applications.

In one aspect of the invention, a skin treatment system is provided comprising a hand-held device having a surface configured to be brought into contact with the skin for transmitting energy to the skin and a coupling emulsion for providing a lubricious surface between the device surface and the skin. The device is typically a hand held ultrasound device but may be any handheld device that transmits energy to the skin. The coupling emulsion may be, for example, a water-in-oil emulsion or and oil-in-water emulsion, and will usually comprise an aqueous phase, an oil phase, and an emulsifier for stabilizing the emulsion and the oil phase may comprise hydrocarbon oils, ester oils, silicone oils, or the like.

Because the use of the device on the skin imparts energy, e.g., heat, sound, etc., an increase in the temperature of the skin occurs during use of the device with concomitant evaporation of volatile solvents that may be present in the emulsion during the use period. Additionally, use of the device imparts shear and downward pressure on the coupling emulsion during use, which may affect the physical properties, in particular the rheology of the emulsion. Loss of volatiles from emulsions lacking the rheology described herein, which generally contain adjuvants stated herein to be limited in the emulsions of the present invention, combined with shear and heat lead to formation of small balls or pills, which can include nonvolatiles, the limited adjuvants, and skin debris (dirt, cellular matter, etc.), which can compromise the aesthetics of the product and which the consumer may find unattractive and may perceive negatively. As used herein, the terms “pilling” or “balling,” refer to a phase separation of the formulation resulting in the formation of small solid deposits on the skin. These small solid deposits are the result of the increase in relative concentration and agglomeration of solid components of the formulation, which increase in relative concentration as volatile components are lost due to evaporation and other liquid components are lost due to absorption into the skin. Soluble components ultimately precipitate out of the composition the emulsion loses solvent. Mechanical friction and rubbing can combine these components with dead skin cells and other skin debris making the pilling even worse. By substantially no pilling or balling is meant that there is only minimal formation of solids during use of the device with the emulsion, which amount, while possibly visible on close inspection, will not cause the user of the device to feel discomfort (e.g., a gritty sensation) as the device moves on the surface of the skin of the user.

The coupling emulsion of the invention is formulated to provide a rheology that substantially eliminates or completely eliminates pilling and balling during normal use. It has been found that coupling emulsions characterized by a rheology where the yield stress value does not substantially increase when stressed are suitable. The yield stress values of the emulsion may vary with stresses placed on it by use of the device, such variations being set forth in the guidelines below. While it is believed that the rheology of the emulsion as described herein exists during the period of use, the measurement of the yield stress at any particular point during the use is not feasible. However, it has been determined that an acceptable coupling emulsion composition can generally be predicted by measuring the yield stress value of the coupling emulsion composition that has been “stressed”, both the stressing and measurement protocols being described hereinafter. Though difficult to measure the rheology of the composition during use, due to the different conditions of use, nevertheless it is believed that the rheology is important to reducing pilling and balling and that the rheology, as measured in accordance with protocols described herein, is predictive of this desired reduction.

In some embodiments, this desirable rheology is maintained notwithstanding evaporation of some or all of the volatile solvents from the starting composition during use of the product as contemplated herein, which will typically be at least two minutes, and more particularly, at least five minutes. In some embodiments, the coupling emulsion has a rheology characterized by a yield stress value that does not increase by more than 50% or, more preferably, remains constant or decreases when the emulsion is stressed, i.e., to give a “stressed” emulsion, as described herein. Due to the special rheology of the emulsion, there is substantially no pilling or balling and preferably no pilling or balling of the emulsion during normal use.

In some embodiments, the rheology is achieved by maintaining the collective weight of all particulate materials in the emulsion at less than 1% by weight of the emulsion and the collective weight of all polymeric film formers at less than 2% by weight of said emulsion. In one embodiment, the emulsion is a water-in-silicone emulsion comprising, as a component of the oil phase, a non-volatile silicone fluid having a viscosity of greater than about 5 centistokes at 37° C. The emulsion may also comprise one or more active ingredients for providing a therapeutic or cosmetic benefit to the skin, and in particular, may comprise active ingredients whose benefits are enhanced in combination with the applied energy, such as ultrasound.

In another aspect, a kit is provided comprising written instructions for using any of the coupling emulsions of the invention to provide a lubricious surface between the skin and the surface of a hand-held device configured to be brought into contact with the skin for transmitting energy to the skin surface. The kit may include such written instructions in combination with a packaged quantity of the emulsion, or in combination with the handheld device, or both.

Also provided is a method for treating the skin comprising applying to the skin a coupling emulsion according to the invention and contacting the skin with the surface of a hand-held ultrasound device to transmit ultrasonic signals into said skin. The treatment area may be, without limitation, the skin of the face, forehead, cheeks, neck, chest, hands, arms, legs, or the like. In some embodiments, the device is applied directly to an area of skin suffering from fine lines and/or wrinkles and/or discoloration, including without limitation areas of hyper-pigmentation known as age spots. The method may be repeated daily for a period of time sufficient to reduce the average wrinkle depth in the skin area or for a period sufficient to reduce discoloration or hyper-pigmentation in the skin area. In some embodiments, the emulsion comprises and active ingredient for improving the appearance of skin. The device may be, for example, a hand-held ultrasound device which transmits ultrasonic energy into said skin.

Typically, the emulsion according to the invention has a yield stress of between about 50-300 Pa, and more typically the yield stress is about 50-250 Pa, and especially from about 50-100 Pa as a fresh or initial composition as well as under conditions of normal use, which are measured as previously described using a stressed emulsion.

Preferably, the yield stress of the coupling emulsion, i.e., the stress at which the elastic modulus equals the viscous modulus or, put another way, the stress at which tan(delta)=1, does not significantly change after exposure to friction or heat. In some embodiments, the yield stress of the emulsion increases by less than a factor of five, preferably less than a factor of four, or less than a factor of three, or even less than a factor of two, when the emulsion undergoes stress. In some embodiments, the stress at which tan(delta)=1 does not increase and can decrease when the emulsion undergoes stress. In some embodiments the yield stress increases by less than 50%, less than 40%, less than 30%, less than 20% or less than 10%, when the emulsion undergoes stress. In some embodiments, the stress at which tan(delta)=1 is decreased, and in certain embodiments decreased by about 10%, by about 50%, or by about 75%, when the emulsion undergoes stress. “Stress” as used in this paragraph means the stressing of an emulsion sample as described herein, which, as explained above, is a means to predict how the emulsion will behave during conditions of normal use of the skin treatment device, e.g., typically be at least two minutes, and more particularly, at least five minutes, wherein evaporation of some or all of the volatile solvents from the fresh or starting composition may occur.

In preferred embodiments, the yield stress after evaporation of some or all of the volatile solvents from the fresh or starting composition, e.g., during use of the product as contemplated herein, remains below about 400 Pa, below about 300 Pa, below about 250 Pa, below about 200 Pa, or below about 150 Pa. In certain particularly preferred embodiments, the emulsion composition has a yield stress that remains substantially below 250 Pa, even after evaporation of some or all of the volatile solvents from the composition, e.g., at the end of use of the product as contemplated herein. It is to be appreciated that the emulsion composition may have a starting yield stress at or below a preferred value, such as any one of those listed above. Where the yield stress is at or close to a preferred value, the yield stress preferably remains essentially unchanged, does not increase, or does not increase substantially, during use of the product, as contemplated herein, so that the yield stress value at the end of use remains below, close to, or at the preferred value. Where the yield stress is considerably below a preferred value, the yield stress may increase and may increase substantially, as long as the yield stress value at the end of use, as contemplated herein, remains below, close to, or at the preferred value. Preferably, the yield stress stays within a certain range during use, as contemplated herein, such that the emulsion composition has and maintains an aesthetically pleasing feel.

The emulsion ideally avoids pilling and balling when exposed to heat or friction, in particular, when exposed to a temperature of 35° C. or more during use of the product as contemplated herein, which will typically be for at least 2 minutes, and more typically for at least five minutes, notwithstanding evaporation of some or all of the volatile solvents from the starting composition. The emulsion preferably is capable of undergoing repeated shear cycles without substantial pilling or balling after the solvents have been substantially removed by evaporation, and preferably when the compositions comprise less than 5%, less than 3%, or than 1% volatile solvents.

Generally, the coupling emulsion is designed to be topically applied to skin and to lubricate and reduce friction between the skin and the probe. Typically, the coupling emulsion allows energy, and particularly ultrasound waves, to be effectively delivered to the tissue. The coupling emulsion can be either a water-in-oil or an oil-in-water emulsion or the like, but typically is an oil-in-water emulsion. The emulsion can have a range of consistencies, from a cream to a serum and may include suitable cosmetic and dermatological excipients and actives.

In some embodiments, the coupling emulsion of the invention includes at least one emollient, and includes a limited amount, in the aggregate, or substantially no insoluble powder or polymer. The aggregate weight percentage of any powder or polymer in the emulsion is typically less than 5% based on the entire weight of the composition, or less than 2.5%, or less than 1%. It has been found, after careful study, that the pilling or balling phenomenon is exacerbated by the presence of significant proportions of polymers or insoluble powders, and particularly when there is a combination of polymers and insoluble powders. Powders, in particular when combined with polymers, provide a dough-like consistency when subject to heat and friction for more than two minutes. Therefore, when powders are included in the composition the concentration of powder is typically less than 1% of the composition, and more typically less than 0.5% of the composition. Typically, the aggregate weight percentage of all powder and polymer constituents in the coupling emulsion is less than 1%, typically less than 0.5%, more typically less than 0.4%, or less than 0.3% or less than 0.2% or less than 0.1%, based on the entire weight of the emulsion. Generally, the emulsion does not contain any abrasive constituents, and preferably contains no particles having an average particle size greater than 20 μm, and preferably contains no particles having an average particle size greater than 10 μm, or more preferably no particles having an average particle size greater than 5 μm in diameter, or greater than 1 μm.

The emollient can be, for example, a silicone fluid, and most often a silicone with reduced volatility and in particular a nonvolatile silicone such as a non-volatile dimethicone. The silicone fluid will generally have a viscosity of greater than about 5 centistokes at 37° C. The emulsion is typically substantially five of a silicone elastomer. Typically, volatile solvents, as hereinafter defined, are limited in the composition and may, in the aggregate, comprise less than 5%, less than 2%, or less than 1% by weight of the emulsion.

The coupling emulsion can include one or more film formers. However, the emulsion typically includes less than five, less than four, or less than three of such film formers. Typically the total film forming material is limited in the emulsion, and total film formers are typically present, in the aggregate, at less than 2%, less than 1%, or less than 0.5% based on the entire weight of the emulsion. If a particulate or powder is also present in the emulsion, then it may be desirable to decrease the amount of the film former to be less than 0.5%, or less than 0.25%, or to omit the film former altogether. Hydrophobic film formers may be present in limited amount and may include, for example, (alkyl)acrylates, polyurethanes, fluoropolymers, silicones, or a copolymer of two or more blocks selected from styrene, alkylstyrene, ethylenelbutylene, ethylene/propylene, butadiene, isoprene, acrylate, and methacrylate.

When the coupling emulsion contains at least one polymer, it may also include a plasticizer. The plasticizer helps to keep the polymer flexible, and helps prevent it from forming a dry brittle film during usage of the coupling emulsion. Typical concentrations would be between 1-10%. Examples of suitable plasticizers include humectants such as glycols, glycerin, and polyethylene glycols, e.g., polyethylene glycols that are liquid at room temperature.

In certain embodiments, the coupling emulsion further contains agents that are delivered to the patient's body during the emission of energy from the probe. The coupling emulsion can comprise one or more anti-glycation agent, one or more anti-oxidants, a dermatologically acceptable excipient or excipients and optionally one or more substance capable of inducing expression of a molecular chaperone.

### BRIEF DESCRIPTION OF FIGURES

FIG. 1 presents representative plots of stress sweeps of fresh (unstressed) (top graph) versus stressed (bottom graph) compositions for sample (A). Elastic Modulus, G′(□); Viscous Modulus, G″(O); Tan(delta) (Δ). Filled and empty symbols indicate separate trials.

FIG. 2 presents representative plots of stress sweeps of fresh (unstressed) (top graph) versus stressed (bottom graph) compositions for sample (C). Elastic Modulus, G′(□); Viscous Modulus, G″(O); Tan(delta) (Δ). Filled and empty symbols indicate separate trials.

FIG. 3 presents summary graphs of stress at Tan(delta)=1 or when G′, G″ cross-over for combined data from experiments on four compositions, two of which wilt pill (A) and (B) and two of which will not pill (C) and (D). FIG. 3(a) is a summary graph of experiments on “fresh” compositions at 25° C. and a gap of 500 μm and FIG. 3(b) is a summary graph showing both data from FIG. 3(a) and results of experiments on “stressed” compositions in which volatile components have been reduced at 37° C. and a gap of 10 μm.

### DETAILED DESCRIPTION

The present invention provides systems and compositions that allow the use of ultrasound or similar techniques for cosmetic applications while avoiding the unpleasant feel of water-based gels and while avoiding the pilling or balling that occurs when a typical cosmetic emulsion is subjected to extended heat or friction. It has surprisingly been found that emulsions with certain rheological characteristics are resistant to pilling or balling and are therefore particularly useful in conjunction with elements that cause friction during cosmetic applications.

### I. DEFINITIONS

As used herein, “percent of a composition,” “percent by weight” or “% wt” refers to the weight percent of the total formulation after addition of any carriers, solvents, emollients, or other components added before application to the skin, unless otherwise specified. All such weights as they pertain to listed ingredients are based on the active level and, therefore, do not include carriers or by-products that may be included in commercially available materials, unless otherwise specifically noted.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

As used herein, the terms “pilling” or “balling,” refer to a phase separation of the formulation resulting in the formation of small solid deposits on the skin. These small solid deposits are the result of the increase in relative concentration and agglomeration of solid components of the formulation, which increase in relative concentration as volatile components are lost due to evaporation and other liquid components are lost due to absorption into the skin As soluble components lose their solvent, these ultimately precipitate out of the composition. Mechanical friction and rubbing can combine these with dead skin cells and other skin debris making the pilling even worse.

When a component is said to be “limited” in the composition as described herein, the component is found in the composition at a concentration of less than about 5%, typically less than about 2.5%, or less than about 1%, or less than about 0.5% by weight of the one or more materials. To the extent that a component that is limited is included at all in the composition, the component will generally comprise from about 5% to about 0.01% by weight of the composition and more typically will comprise from about 2% to about 0.05%, and typically from about 1% to about 0.25% by weight of the composition.

A composition described herein is said to be “substantially free” of a component if the component is present at such low levels as to not have a measurable input on theology, particular on balling and pilling, of the total composition. Typically, a composition is described as “substantially free” of a component when the component comprises less than about 1% by weight of the composition, more typically, less than about 0.5% of the composition, and most typically the component is absent from the composition. The compounds will generally comprise from about 0% to about 1% by weight of the composition and more typically will comprise from about 0% to about 0.5%, and typically from about 0% to about 0.05%, from about 0% to about 0.01%, or from about 0% to about 0.001% by weight of the composition.

For purposes of this disclosure, a “yield stress” of a composition is defined as the stress value at which the elastic modulus (G′) is equivalent to the viscous modulus (G″) (i.e. when tan(delta)=1) when the sample is measured as follows:

In an AR-G2 Rheometer (TA Instruments) equipped with parallel plate geometries (top plate of 40 mm in diameter operated at a gap of 500 um for “fresh samples”, e.g., at 25° C.

The top plate is oscillated at a frequency of 1 s⁻¹ as the applied oscillator stress is gradually increased. The increasing stress results in increasing strain or deformation and the rheological properties (i.e. G′, G″, tan(delta)) are measured using standard techniques. The elastic modulus (G′) is directly correlated to the stiffness of the sample while the viscous modulus (G″) accounts for the liquid-like component. The tan(delta) or tangent of the phase angle is equal to the ratio of G″ to G′. The tan(delta) is a measure of how structured the sample is (i.e., tan(delta)<1 for solid-like and tan(delta)>1 for liquid-like samples). Therefore, the transitions of the sample from a solid-like material to a liquid-like material is marked by when tan(delta)=1.

For the purpose of this disclosure, “fresh” sample is defined by a composition that comes from a sealed container stored at ambient conditions immediately upon opening the container and immediately upon exposure of the composition to ambient conditions, and prior to measurement.

A “stressed” sample is one that has undergone the steps of a procedure for treating the sample as follows:
A 40 mil (1.02 mm in thickness) wet film of the sample is placed on the bottom plate of the AR-G2 Rheometer, which is also equipped with a peltier heating element.
   The sample is then heated at 50° C. for 30 minutes and then cooled to 37° C. prior to the measurements.
   The top plate is then lowered to create a 10-μm gap between the plates and the rheological properties measured as described above. The top plate used in these measurements is 20 mm in diameter and serrated to prevent the sample from slipping.

### II. METHODS OF USE

A method for non-ablative treatment of skin is provided to improve skin quality by applying a coupling emulsion to an area of skin and delivering friction and/or energy, such as an ultrasound wave, to the skin. Improvements in skin quality can include enhancing the elasticity of the skin, improvements in texture, as measured by softer skin or reduced pore size or increased skin resilience, reduction in skin sagging and atrophy, improvements in signs of aging such as maintenance of skin integrity and reduced skin thinning, and reduction in signs of fine lines and wrinkles, or improvement in skin tone and coloration such as reduced blotchiness or sun damage. Typically, the subject receiving the treatment is in need of at least one improvement described above. In certain embodiments, the methods can deliver an active ingredient that is incorporated in the emulsion to the surface layers of the skin.

In an exemplary embodiment, focused, unfocused or defocused energy is applied to a region of interest on a subject to elicit a biochemical or biophysical response resulting in an improvement in skin quality. Any device that increases friction, emits or conducts ultrasound, light, heat, electric energy, or any other type of energy, including mechanical energy such a vibration, rotation or pulsation, that may provide a beneficial or sensorally pleasing effect to the local area can be used in conjunction with the coupling emulsion described herein. However, typically, the device includes a probe to apply ultrasound energy to the region. Typically, the ultrasound is non-focused to improve skin quality without need for a professional application, however in other embodiments, the ultrasound can be focused, Cosmetic treatments can be hindered by the barrier function of the epidermis and in particular the outer stratum corneum. The epidermis provides a significant mechanical and chemical barrier to solute transfer due to the cornified bilayer. Also, there is significant enzymatic activity in the epidermis and dermis, which provides a biochemical defense to neutralize applied xenobiotics and which is comparable to that of the liver in terms of activity per unit volume. Additionally, the molecular weight of active substances is known to be important in determining their propensity to diffuse across the skin. Diffusion of substances of molecular weight around 500 Da and above is known to be inefficient.

Wrinkles are generally a result of the natural aging process of the skin, and of exposure to the sun's ultraviolet rays. A wrinkle is a configuration change in the surface of the skin Generally, wrinkles are classified as described in Kligman et al. (1985) Br J Derm 113:37-42. Kligman classifies wrinkles into three classes: linear wrinkles, glyphic wrinkles, and crinkles. Linear wrinkles are straight, found generally in the facial skin, and are caused by natural aging or exposure to ultraviolet light. Glyphic wrinkles are shaped as apparent triangles or rectangles of wrinkles, are found on the face, hands, and neck exposed to sunlight, and are aggravated by exposure to ultraviolet light or dermatoheliosis. Crinkles are thin, crinkled wrinkles on flabby skin, found anywhere on the skin, but typically on the backs of hands and around the eyelids.

In certain embodiments, the coupling emulsions can be administered in conjunction with application of friction. Typically, the friction is combined with delivery of energy, such as ultrasound energy, and can be combined with heating for: (a) treatment, reduction, and/or prevention of fine lines or wrinkles; (b) reduction of skin pore size, (c) improvement in skin thickness, plumpness, and/or tautness; (d) improvement in skin suppleness and/or softness; (e) improvement in skin tone, radiance, and/or clarity; (f) improvement in procollagen and/or collagen production; (g) improvement in maintenance and remodeling of elastin; (h) improvement in skin texture and/or promotion of retexturization; (i) improvement in skin barrier repair and/or function; (j) improvement in appearance of skin contours; (k) restoration of skin luster and/or brightness; (l) replenishment of essential nutrients and/or constituents in the skin; (m) improvement of skin appearance decreased by menopause; (n) improvement in skin moisture; or (o) increase in skin elasticity and/or resiliency.

The effect of a coupling emulsion on the formation or appearance of wrinkles can be evaluated qualitatively, e.g., by visual inspection, or quantitatively, e.g., by microscopic or computer assisted measurements of wrinkle morphology or skin topography. Typically, wrinkle morphology is quantitatively analyzed, e.g., the number, depth, length, area, volume and/or width of wrinkles per unit area of skin, surface roughness of wrinkle area or height distribution of wrinkle area are measured, Examples of quantitative methods for measuring wrinkles include, but are not limited to, the optical cut technique employing a laser beam, as proposed by Hoshino (1992) Pixel 45:121, herein incorporated by reference; or methods which analyze three-dimensional skin replicas, e.g., the Shiseido Wrinkle Analyzer 3D Pro system (Takasu et al. (1996) J Soc Cosmet Chem Japan 29:394 405). Another method of measuring the 3D profile of human skin is the PRIMOS optical 3D in vivo skin measurement device (GFMesstechnik, Teltow, Germany). The skin damage that can be improved or treated with the emulsions of the invention include any signs of reduced skin elasticity such as fine lines and/or wrinkles, fragile or thinning skin, sagging skin, lack-luster skin, fatigued skin, dry skin, skin sensitivity, dark eye circles, puffy skin, irregular skin pigmentation, and melasma.

Topically applying emulsions of the present invention to the skin can enhance and improve the aesthetic appearance of skin by, among other improvements, decreasing skin fragility; preventing and reversing deterioration of elastin; preventing skin atrophy; promoting/accelerating cell turnover; improving skin firmness/plumpness; improving skin texture; decreasing fine lines and wrinkles; improving skin tone; enhancing skin thickness; restoring skin luster; minimizing signs of fatigue; reducing skin dryness; reducing skin itchiness; reducing skin redness; reducing sensitivity to chemical, mechanical or radiation impact; reducing propensity of the skin to flush and blush; reducing dark circles and puffiness in the periorbital eye area; reducing frown lines on the forehead and laugh lines around the mouth; increasing cell proliferation; decreasing the extent and/or duration of bruising visible after physical trauma; reducing blotchiness and irregular skin pigmentation; treating or ameliorating melasma; treating or ameliorating skin hyperpigmentation; treating or ameliorating foliculitis barbae and ingrown hair bumps and after-shave nicks and irritation; treating or ameliorating dermatitis, psoriasis and other skin conditions affiliated with or caused by acute, subacute or subchronic inflammation; and enhancing overall skin health.

The invention also provides a method for treating aging skin by topically applying a coupling emulsion comprising over the affected area for a period of time sufficient to reduce, ameliorate, dermatological signs of reduced skin elasticity.

In an exemplary method, the system is configured to apply energy to an area of the skin. In specific embodiments, the system includes a probe, configured to be in contact with the emulsion, that provides an unfocused energy to skin through the emulsion. Typically, the system includes a probe that delivers ultrasound energy to the skin. Generally, the probe is configured to provide low intensity energy, and particularly low intensity ultrasound, to the skin.

In some embodiments, the system is configured so that temperature in a region of interest approximately 0.1-10 millimeters below the surface of the patient's skin is increased by less than 5° C., or less than or less than 3° C., or less than 2° C., or less than 1° C., by applying unfocused or defocused ultrasound energy to the region of interest. In one exemplary embodiment ultrasound energy is applied at known depths over an extended area without initial or ongoing imaging.

In an exemplary embodiment, the energy is applied to the surface, or approximately 0.1-5 millimeters below the surface of the patient's skin and raises the temperature at this depth in a range of approximately 1-10° C. higher than the patient's normal body temperature and causes certain mechanical effects at the region of interest. In an exemplary embodiment, the temperature increase is between 1-5° C. higher than the patient's normal body temperature. Typically, the heating occurs below the surface of the skin. Therefore, the temperature at the specific depths in the region of interest is typically approximately 35-49° C. during the therapy.

In an exemplary embodiment, enough energy is emitted from the ultrasound system to stay below the thermal capacity of the tissue. Therefore, no ablation or coagulation of the tissue occurs.

In an exemplary embodiment, the heat causes increased blood perfusion in the region of interest. Additionally, the heat raises the temperature to a level where proteins within the region of interest are denatured. Further, heat can initiate programmed cell death or apoptosis of bacteria cells that contribute to acne.

In an exemplary embodiment treatment is used to suppress the activity of sebaceous glands, thereby reducing the size and number of skin pores, decreasing skin oiliness, and achieving a desirable cosmetic effect. Typically the method is a method of cosmetic treatment of cosmetic skin conditions. However the invention also encompasses the treatment of medical skin conditions, in which instances the method is a method of medical treatment.

A method for treatment of the skin may further comprise application of ultrasound directly or indirectly to an area of skin to which the emulsion has been applied, or as a pre-treatment to an area of skin to which the emulsion is to be applied.

In certain methods of the invention application of ultrasound is performed at low and/or high frequency directly or indirectly to an area of the skin where the emulsion has been applied, or is to be applied. Low and high frequency ultrasound can be applied simultaneously, sequentially or separately, e.g. sequentially as several alternating single applications of low and high frequency or, separately where a series of applications of low frequency is alternated with a series of applications of high frequency. Low frequency ultrasound is believed to be useful to facilitate delivery of molecules to the skin (a process termed “sonophoresis”). High frequency ultrasound has a lesser sonophoretic effect than low frequency, but it also has many other effects beneficial to the skin in that it stimulates fibroblast proliferation, stimulates collagen and other extracellular matrix (ECM) component formation (e.g. fibrillin), stimulates blood supply, renews the elastic quality of ECM which stiffen with age, stimulates the expression of Heat Shock Proteins (HSPs—intracellular molecular chaperones) in fibroblasts (dermis) and keratinocytes (epidermis) through thermal and mechanical stimulation.

In methods involving application of ultrasound, a low frequency component of the ultrasound is typically applied in continuous mode and, in the event that any high frequency component is included, the high frequency component is typically applied in pulsed mode.

The term “ultrasound” describes sound frequencies of 20 kHz and above, a low ultrasound frequency is from 20 to 500 kHz, the spatial average power density of the low frequency ultrasound energy being from 20 to 500 mW/cm²; a high ultrasound frequency from 500 kHz (0.5 MHz) to 3.5 MHz, the spatial average power density of the high frequency ultrasound energy being from 0.005 to 5 W/cm², more typically, 0.01 to 2 W/cm², and even more typically 0.02 to 1 W/cm².

Typically, the area of the skin that is being targeted by a probe is targeted for at least 2 minutes. More typically, the skin area is targeted for at least four minutes or at least five minutes or at least six minutes or at least seven minutes or at least 8 minutes. In certain embodiments, the area is targeted for less than ten minutes. In certain embodiments, the region of the subject, for example the face, is targeted with ultrasound treatment through a hand-held probe for between 2-8 minutes, more typically between 4-8 minutes, especially 5-7 minutes.

The ultrasound can be applied using a hand-held probe, optionally adapted for application of the coupling emulsion to the skin. For example, a cartridge/dispenser can be attached to the ultrasound probe such that the formulation is gradually released as the probe is moved around the skin surface, the cartridge may contain a pre-set amount of formulation. Different cartridges with different formulations can be attached depending on the skin condition being treated, e.g. different cartridges may contain different compositions for anti-ageing treatments, the treatment of scars, stretch-marked skin or cellulite. The ultrasound is applied by gently massaging the probe on the skin in a circular or linear stroking movement.

Also provided is a kit comprising a coupling emulsion according to the invention and optionally, a device comprising an ultrasound source and/or optionally a probe for applying ultrasound to the skin and/or for applying the emulsion to the skin. A kit according to the invention is suitable for performing a method of the invention as described herein. A kit may further comprise instructions for use of the kit.

The invention provides the use of a coupling emulsion in the treatment of a skin condition and in particular a cosmetic skin condition. The skin conditions can be selected from the group: “orange peel” skin appearance, abnormal desquamation (or exfoliation) or abnormal epidermal differentiation (e.g. abnormal skin turnover) such as scaliness, acne and acne scars, alterations to underlying tissues (e.g. subcutaneous fat, cellulite, muscles, trabeculae, septae, and the like), atrophy such as that associated with ageing or steroid use, blemishes, botching (e.g. uneven red coloration due to, e.g. rosacea), bumps, chapping, coarseness, collagen breakdown and structural alterations or abnormalities and discolorations (e.g. changes in the stratum corneum, dermis, epidermis, the skin vascular system such as tellangiectasia), crevices, dermatohetiosis, dryness and dry skin conditions, excess skin oil problems such as over-production of sebum, expression lines, facial shine or oiliness, flakiness and/or other forms of skin unevenness or roughness, foundation breakthrough, hair loss, hyperkeratinization, inadequate skin moisture (or hydration) such as caused by skin barrier damage, irregular pigmentation, keratosis, large pores (e, g, associated with adnexal structures such as sweat gland ducts, sebaceous glands, or hair follicles), loss of skin elasticity (loss and/or inactivation of functional skin elastin and loss of skin recoil from deformation) such as elastosis, firmness and/or tightness, melanin-related hyper-pigmented (or unevenly pigmented) skin regions and non-melanin skin discoloration such as under-eye circles, other histological or microscopic alterations in skin components such as ground substance (e.g. hyaluronic acid, glycosaminoglycans, etc.), photodamage, post-inflammatory hyper-pigmentation such as that which occurs following an inflammatory event (e.g. an acne lesion, in-grown hair, insect/spider bite or sting, scratch, cut, wound, abrasion, and the like), rashes, rhytides, sagging (including puffiness in the eye area and jowls), sallowness (pale color), scaliness, scarring (including hypertrophic and keloid scars), stretch marks, tissue responses to insult such as itch or pruritus, wrinkles (including both fine superficial wrinkles and coarse deep wrinkles).

The invention also provides the use of a coupling emulsion in a medical treatment or as a medicament. Further provided is the use of a composition according to the invention in the manufacture of a medicament for the treatment of a medical skin conditions such as those described above.

### III. SYSTEMS

A system for non-ablative treatment of skin is provided. In an exemplary embodiment a system is provided including a device configured to apply friction and, typically energy such as ultrasound energy, to a region of interest on a subject and a coupling emulsion formulated to be applied to skin and to lubricate the area of the skin to reduce friction with the probe. The emulsion with the rheological properties described herein avoids pilling and balling when exposed to friction.

Typically, the emulsion is formulated to avoid pilling or balling during use of the device, i.e., when it is exposed to a temperature at least 35° C. for at least 2 minutes, or at least five minutes, notwithstanding evaporation of some or all of the volatile solvents from the starting composition during the contemplated use. Typically, the emulsion has a yield stress of between about 50-300 Pa and more typically, the yield stress is about 50-100 Pa. Typically, the stress at which tan(delta)=1 of the emulsion does not increase by more than 20%, or more than 10%, or more than 5% when exposed to a temperature of at least 35° C. for at least 2 minutes.

An exemplary system is configured to provide energy to a region of interest through the coupling emulsion. In general, the energy is provided to a region of interest by applying unfocused or defocused ultrasound energy. In one exemplary embodiment ultrasound energy can be applied at known depths, for example about 0.1 to about 10 millimeters below the surface of the skin over an extended area without initial or ongoing imaging. In an exemplary embodiment, the device is configured to emit energy to stay below the thermal capacity of the tissue.

In one embodiment, a system is provided that comprises an ultrasound system that emits ultrasound energy at concentrated levels to the region of interest at specific or targeted area and a coupling emulsion that is a silicon-in-water emulsion.

In an exemplary embodiment, a coupling emulsion is used to acoustically couple the probe to a patient's body. In certain embodiments, the coupling emulsion further contains agents that are delivered to the patient's body during the emission of energy from the probe.

An exemplary ultrasound device comprises a control system, a probe, and a display or indicator system. The probe can comprise various probe and/or transducer configurations. In an exemplary embodiment, the probe delivers unfocused ultrasound energy to the region of interest without performing an imaging function. In other exemplary embodiments, the probe delivers strongly focused or weakly focused ultrasound energy. In yet other exemplary embodiments, imaging can be completed during treatment. In other exemplary embodiments, the probe can be configured for a combined dual-mode imaging/therapy transducer, coupled or co-housed imaging/therapy transducers, or simply a therapy probe or an imaging probe. Most typically, a probe is designed to be used by a subject in an ‘at home’ setting.

The control system and display system can also comprise various configurations for controlling probe and system functionality, including for example a microprocessor with software and a plurality of input/output devices, a system for controlling electronic and/or mechanical scanning and/or multiplexing of transducers, a system for power delivery, systems for monitoring, systems for sensing the spatial position of the probe and/or transducers, and systems for handling user input and recording treatment results, among others.

The ultrasound applying apparatus employed generally includes a housing with a probe for applying the ultrasound to the user's skin, and a driver circuit that provides an electric pulse (signal) for actuating the probe to transmit the ultrasound to the skin. The probe is composed of a piezoelectric element generating the ultrasound, a probe head that includes a mounting face and an opposing face for use in contact with the skin. The delivery probe carries the piezoelectric element to transmit the ultrasound to the skin. The probe head generally resonates with the electric signal from the driver circuit, thereby transmitting resulting vibrations to the skin. The combined vibration mass gives a first electrically equivalent impedance when it is normally loaded by contact with the skin, and gives a second electrically equivalent impedance when it is unloaded. The apparatus includes a load detecting circuit which monitors whether the combined vibration mass give the first or second electrically equivalent impedance and provides a load detection signal only upon seeing the first electrically equivalent impedance.

Also included in the apparatus is a control circuit which limits or stops the electric pulse when the load detection signal is not received within a predetermined time period. The combined vibration mass has a structure that restrains vibrations at a center portion of the vibration mass in order to reduce a parasitic resonance, thereby differentiating the first electrically equivalent impedance from the second electrically equivalent impedance. Thus, the load detecting circuit can successfully judge whether the probe head is in contact with or out of contact from the skin, whereby the control circuit can be made reliable to limit the ultrasonic vibrations from being generated when the probe is unloaded.

The control circuit is designed to receive the first electrically equivalent impedance and constitute the control element that varies the intensity of the ultrasound generated at the vibrator element in accordance with the magnitude of the first electrically equivalent impedance. As the first electrically equivalent impedance will vary depending upon a pressure at which the horn or the combined vibration mass is held against the user's skin, the device can vary the effect or the strength of the ultrasound being applied to the skin depending upon the pressure, thereby applying the ultrasound optimally to the user's skin for enhanced skin care result.

Typically, the ultrasound probe is a hand-held probe, optionally adapted for application of an emulsion according to the invention to the skin. In certain embodiments, a cartridge/dispenser can be attached to the probe adapted to release the coupling emulsion gradually as the probe is moved around the skirt surface. The cartridge may contain a pre-set amount of formulation. Different cartridges with different formulations can be attached depending on the skin condition being treated, e.g. different cartridges may contain different compositions for anti-ageing treatments, the treatment of scars, stretch-marked skin or cellulite. The ultrasound is applied by gently massaging the probe on the skin in a circular or linear stroking movement.

The apparatus typically includes a motion detecting circuit which monitors whether the combined vibration mass is moving and provides a motion detection signal when the vibration mass is so moving. The control circuit is connected to receive the motion detection signal and controls the driver circuit to stop or limit the electric pulse when the motion detection signal is not continuous over a critical time duration even in the presence of the load detection signal being detected within the predetermined time period.

In other embodiments, the ultrasound device is not adapted to deliver the coupling emulsion and the emulsion is provided separately in a container designed to deliver an appropriate amount of emulsion for a ‘single use’. Typically, the container is a pump and most typically it is configured to deliver between 1 and 20 cc of coupling emulsion, more typically between 1 and 10 and most typically between about 1 and 5 cc, or about 1 or about 2 or about 3 or about 4 or about 5 cc of coupling emulsion.

Any amount of energy can be used during method as long as the tissue within is not ablated or coagulated. In an exemplary embodiment, the energy emitted from probe is unfocused or defocused ultrasound energy. Alternatively, focused ultrasound energy could be emitted from and applied to the skin.

in certain exemplary embodiments, system is equipped with certain features to aid the user. One feature is a disposable tip that covers the probe during use. The disposable tip enables ultrasound energy to pass through the tip and contact the patient. But, the disposable tip can be removed from probe after use and replaced with a new disposable tip to prevent the spread of germs from one patient to another that might reside on probe after contact with a patient's skin. Different size disposable tips can be used and fall within the scope of the present invention.

In one exemplary embodiment, the energy released into the area does not increase local temperature in the tissue. In other embodiments, the energy released increases the local temperature less than approximately 25° C. over a body's normal temperature. The temperature within the area being treated is typically between approximately 35-60° C. In another exemplary embodiment, the temperature is raised approximately 1-15° C. over a body's normal temperature, or about 1-10° C., or less than 10° C., such as less than 5° C., or less than 1° C. Typically, the temperature of skin in the vicinity of the element providing friction and energy is between approximately 35-49° C.

Generally, any conventional system that provides friction in an area can be used. Most typically, energy is also provided to improve skin texture or related properties. Typically, conventional ultrasound systems are used. Typically, the ultrasound system includes a component to generate the ultrasonic waves and a probe connected to the component, wherein the probe is configured to be used on a subject's face. Typically, ultrasound devices are described, for example, in U.S. Pat. Nos. 7,481,781, 6,821,274, 6,461,586, 6,088,613 and Publication Nos. 2009/0163836, 2004/0265393, 2003/0229283 and 2002/0193784.

In certain embodiments, the system includes a self-contained voltage generating system incorporated in a package or device for housing a product. In these embodiments, there is at least one piezoelectric element incorporated in the package or device and the piezoelectric element generates a source of voltage when it is activated. The voltage is then used to perform various activities on other elements that are part of the package or device, such as operate a motor, provide heat, provide ultrasonic energy, furnish light, provide acoustic energy, and provide vibration energy. The piezoelectric elements are in the form of discrete particles, piezoelectric fibers, filaments, transducers, and actuators. U.S. Publication No. 2008/0143214, the disclosure of which is incorporated herein by reference, describes such a device.

### IV. EMULSIONS

The coupling emulsion is formulated to be applied to skin and to lubricate the skin. In some embodiments, a coupling emulsion which is to be applied in conjunction with ultrasound treatment (where the emulsion is applied prior to, during ultrasound treatment, or shortly after an ultrasound pre-treatment), will have a viscous nature, so that a layer of the emulsion can be spread on the skin and will remain in place on the skin until it is removed, e.g. by wiping the emulsion away with tissue or cotton wool, or by rinsing the formulation off. However, it is particularly beneficial for the methods of the invention if the composition remains on the skin for the duration of the treatment, but evaporates or is absorbed into the skin shortly after completion of the treatment. In particular, it is generally envisioned that the emulsion is absorbed into the skin between 8 and 10 minutes after application when thermal energy is applied for between 2 and 8 minutes.

An emulsion according to the invention is typically at a pH close to the pH of skin, e.g. at a pH of from pH 4 to pH 6, or pH 4.5 to pH 5.5. The emulsion may also include AHA formulations which have a pH of between 3.5 and 4.

The coupling emulsions according to the invention are typically formulated as oil-in-water emulsions, These emulsions comprise a water-containing continuous phase and an oil-containing discontinuous phase. The coupling emulsions may also be formulated as water-in-oil emulsions. These emulsions comprise an oil-containing continuous phase and an aqueous discontinuous phase. The emulsions will comprise sufficient amounts of oil and water to make oil-in-water or water-in-oil emulsions.

For oil-in-water emulsions, the aqueous phase will typically comprise from about 10% to about 99%, from about 20% to about 85%, or from about 30% to about 70% by weight, based on the total weight of the emulsion, and the oil-containing phase will typically comprise from about 1% to about 90%, from about 5% to about 70%, or from about 20% to about 60% by weight of the total emulsion. For water-in-oil emulsions, the oil-containing phase will typically comprise from about 10% to about 99%, from about 20% to about 85%, or from about 30% to about 70% by weight, based on the total weight of the emulsion, and the aqueous phase will typically comprise from about 1% to about 90%, from about 5% to about 70%, or from about 20% to about 60% by weight of the total emulsion.

The oil-containing phase may be composed of a single oil or mixtures of different oils. Essentially any oil is contemplated to be useful, although non-volatile highly hydrophobic oils are typical, Suitable non-limiting examples include vegetable oils; esters such as octyl palmitate, isopropyl myristate and isopropyl palmitate; ethers such as dicapryl ether; fatty alcohols such as cetyl alcohol, stearyl alcohol and behenyl alcohol; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; natural or synthetic waxes; and the like.

Suitable hydrophobic hydrocarbon oils may be saturated or unsaturated, have an aliphatic character and be straight or branched chained or contain alicyclic or aromatic rings. Hydrocarbon oils include those having 6-20 carbon atoms, more typically 10-16 carbon atoms, Representative hydrocarbons include decane, dodecane, tetradecane, tridecane, and C₈₋₂₀ isoparaffins. Paraffinic hydrocarbons are available from Exxon under the ISOPARS trademark, and from the Permethyl Corporation. In addition, C₈₋₂₀ paraffinic hydrocarbons such as C₁₂ isoparaffin (isododecane) manufactured by the Permethyl Corporation having the tradename Permethyl 99A™ are also contemplated to be suitable. Hydrocarbon oils include dodecane, isododecane, squalane, hydrogenated polyisobutylene, docosane (i.e., a C₂₂ hydrocarbon), hexadecane, and isohexadecane. Also useful are the C₇₋₄₀ isoparaffins, which are C₇₋₄₀ branched hydrocarbons. Various commercially available C₁₆ isoparaffins, such as isohexadecane (having the tradename Permethyl R™) are also suitable. Examples of volatile hydrocarbons include polydecanes such as isododecane and isodecane, including for example, Permethyl-99A (Presperse Inc.) and the C₇-C₈ through C₁₂-C₁₅ isoparaffins such as the Isopar Series available from Exxon Chemicals.

In certain embodiments, the emulsion comprises an oily component selected from C₁₋₃₀ alcohol esters of C₁₋₃₀ carboxylic acids and of C₂₋₃₀ dicarboxylic acids, hydrocarbon oils, mono-, di- and tri-glycerides of C₁₋₃₀ carboxylic acids, silicone oils, mineral oil and petrolatums, vegetable oils and hydrogenated vegetable oils, animal fats and oils, silicone oils, aromatic oils, and mixtures thereof. Fatty acid esters include cetyl 2-ethylhexyl, isopropyl myristate, myristyl myristate, isopropyl palmitate, cholesterol; more typically cetyl 2-ethylhexyl and myristyl myristate; and triglycerides such as capryticicapric triglyceride, PEG-6 caprylic/capric triglyceride, and PEG-8 caprylic/capric triglyceride, Meadowfoam Seed Oil.

The aqueous phase is typically at least 50% water, such as at least 50%, at least 55%, at least 60%, at least 65%, at least 70% or at least 75% water or more, but may also include one or more additional solvents. The additional solvents are typically primarily non-volatile solvents. Volatile solvents should generally be limited in the emulsion as the heat and friction of the ultrasound will increase evaporation and promote pilling or balling of the emulsion on the skin. The additional solvents will typically comprise from about 0.1% to about 50% by weight of the aqueous phase, more typically up to about 1% by weight, and typically up to about 30% by weight of the aqueous phase.

Typically, additional solvents used in the emulsion do not evaporate substantially and/or significantly for at least 8 minutes when exposed to normal skin temperature. Typically, the solvents evaporate minimally within less than about 8 minutes at 35° C., or at 40° C. or at 45° C. or more. Most typically, the solvent is considered “non-volatile” if 100 uL of the sample do not evaporate when exposed to a temperature of 40° C. for 8 minutes, e.g., when spread on a surface as contemplated with the use of the compositions described herein. Typically, volatile solvents will exhibit a vapor pressure above about 0.01 mmHg at 20° C. In some embodiments, there is little or no “pilling and balling” after evaporation of all solvents having a vapor pressure of greater than 0.02 mmHg at 20° C.; in some embodiments there is little or no “pilling and balling” after evaporation of all solvents having a vapor pressure greater than 0.03 mmHg at 20° C. The solvents will typically have a viscosity of greater than about 5 centistokes, or greater than about 10 centistokes, or greater than about 20 centistokes, or greater than about 30 centistokes, or greater than about 40 centistokes, or greater than about 50 centistokes at 25° C.

### A. Silicone Emulsion

The emulsion can be a silicone-in-water (with silicone as the discontinuous ‘internal’ phase) or a water-in-silicone emulsion (with silicone as the continuous ‘external’ phase) but is most often a silicone-in-water emulsion.

For silicone-in-water emulsions, the silicone-containing phase will typically comprise from about 1% to about 60%, from about 1% to about 50%, or from about 1% to about 20%, or from about 1% to about 15%, or from about 1% to about 10% by weight of the total emulsion. The silicone oil phase will typically comprise about 85 to 100% by weight of one or more suitable silicones by weight of the silicone phase. For water-in-silicone emulsions, the aqueous phase will typically comprise from about 10% to about 90%, or from about 20% to about 80%, from about 30% to about 80%, or from about 40% to about 80%, or from about 40% to about 70% by weight of the total emulsion. The aqueous phase will typically comprise from about 25% to about 100%, more typically from about 50% to about 95% by weight water.

The silicone oil phase typically includes non-volatile silicone oils as an emollient. Typically, these silicone oils will have a viscosity of greater than about 5 centistokes, or greater than about 10 centistokes, or greater than about 20 centistokes, or greater than about 30 centistokes, or greater than about 40 centistokes, or greater than about 50 centistokes at 25° C.

Examples of suitable silicone oils include polyalkylsiloxanes, cyclic polyalkylsiloxanes, and polyalkylarylsiloxartes. Commercially available polyalkylsiloxanes include the polydimethylsiloxanes, non limiting examples of which include dimethyl polysiloxane (dimethicone), phenyl trimethicone, and diphenyldimethicone, polyarylsiloxanes, polyalkylarylsiloxanes, or mixtures thereof. Examples of dimethicones include the Vicasil™ series sold by General Electric Company and the Dow Corning™ 200 series sold by Dow Corning Corporation. Suitable dimethicones include alkyl-substituted dimethicones such as cetyl dimethicone and lauryl dimethicone. Commercially available dimethiconols are typically sold as mixtures with dimethicone or cyclomethicone Dow Corning™ 1501 and 1503 fluids), Commercially available cyclic polyalkylsiloxanes include Dow Corning™ 244 fluid, Dow Corning™ 344 fluid, Dow Corning™ 245, and Dow Corning™ 345 fluid. The silicone oils may optionally be substituted will various functional groups such as alkyl, aryl, amine groups, vinyl, hydroxyl, haloalkyl groups, alkylaryl groups, and acrylate groups. As an alternative, polydiethylsiloxanes (Diethicones) are hybrid silicone polymers with other than methyl substitution. The alkyl trisiloxanes can also be used as these are light, dry, emollient oils with good organic compatibility and include TM-081 Caprylyl Methicone, TM-121 Lauryl Methicone, and TM-181 Stearyl Methicone. Fluorocarbon silicones such as FCS-331, a highly lubricious gel consisting of submicron particles of a tetralltioroethylene/hexafluoropropylene copolymer dispersed in a fluorinated dimethyl fluid are also useful. The gel has the unusual property of increasing slip as higher amounts of shear force are applied, however the fluorinated dimethyl fluid, the base for FCS-331, is insoluble in other polydimethylsitoxane fluids and common organic oils, but can be dispersed in cyclic siloxanes for incorporation into emulsions and anhydrous systems, therefore in these embodiments, the emulsion further comprises a cyclic siloxane.

### B. Emulsifier

A coupling emulsion will typically contain an emulsifier. The amount of emulsifier will typically be from about 0.001 wt % to about 10 wt %, but typically will range from about 0.01 to about 5 wt %, or about 0.1 wt % to about 1 wt %, based upon the total weight of the emulsion. The emulsion is typically emulsified with a nonionic surfactant emulsifier.

Emulsifiers that can be used in the coupling emulsion include, as non-limiting examples: sorbitan esters; polyglyceryl-3-diisostearate; sorbitan monostearate, sorbitan tristearate, sorbitan sesquioleate, sorbitan monooleate; glycerol esters such as glycerol monostearate and glycerol monooleate; polyoxyethylene phenols such as polyoxyethylene octyl phenol and polyoxyethylene nonyl phenol; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2, Steareth-10, and Steareth-20, polydiorganosiloxane-polyoxyalkylene block copolymers, including those described in U.S. Pat. No. 4,122,029, the disclosure of which is hereby incorporated by reference and emulsifiers that are provided in the INCI Ingredient Dictionary and Handbook 11th Edition 2006, the disclosure of which is hereby incorporated by reference. Other suitable water-in-silicone emulsifiers are disclosed in U.S. Pat. No. 6,685,952, the disclosure of which is hereby incorporated by reference herein. Commercially available water-in-silicone emulsifiers include those available from Dow Corning under the trade designations 3225C and 5225C FORMULATION AID; SILICONE SF-1528 available from General Electric; ABM EM 90 and EM 97, available from Goldschmidt Chemical Corporation (Hopewell, Va.); and the SILWET™ series of emulsifiers sold by OSI Specialties (Danbury, Conn.).

Additional examples of emulsifiers include, but are not limited to, dimethicone PEG 10/15 crosspolymer, dimethicone copolyol, cetyl dimethicone copolyol, PEG-15 lauryl dimethicone crosspolymer, laurylmethicone crosspolymer, cyclomethicone and dimethicone copolyol, dimethicone copolyol (and) caprylic/capric triglycerides, polyglyceryl-4 isostearate (and) cetyl dimethicone copolyol (and) hexyl laurate, and dimethicone copolyol (and) cyclopentasiloxane. Specific examples include PEG/PPG-18/18 dimethicone (trade name 5225C, Dow Corning), PEG/PPG-19/19 dimethicone (trade name BY25-337, Dow Corning), Cetyl PEG/PPG-10/1 dimethicone (trade name Abil EM-90, Goldschmidt Chemical Corporation), PEG-12 dimethicone (trade name SF 1288, General Electric), lauryl PEG/PPG-18/18 methicone (trade name 5200 FORMULATION AID, Dow Corning). Emulsifiers with crosslinked silicone monomers such as PEG-10 dimethicone crosspolymer (trade name KSG-20, Shin-Etsu), and dimethicone PEG-10/15 crosspolymer (trade name KSG-210, Shin-Etsu, being somewhat elastonteric in nature, may be suitable as emulsifiers but only at low levels,

### C. Humectant

A humectant is typically added to the emulsion to absorb and retain water. The coupling emulsion generally includes from about 0.1% to about 10% of a humectant, more typically from about 1-10%, or from about 1-8% or from about 2-7%, or from about 3-7% of a humectant.

Water soluble humectants include polyhydric alcohols such as butylene glycol (1,3 butanediol), pentylene glycol (1,2-pentanediol), glycerin, sorbitol, propylene glycol, hexylene glycol, ethoxylated glucose, 1,2-hexane diol, 1,2-pentane diol, hexanetriol, dipropylene glycol, erythritol, trehalose, diglycerin, sorbitol, xylitol, maltitol, maltose, glucose, fructose; and other water-soluble compounds such as sodium chondroitin sulfate, sodium hyaluronate, sodium adenosin phosphate, sodium lactate, pyrrolidone carbonate, glucosamine, cyclodextrin, and mixtures thereof. Other humectants include alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

When the coupling emulsion contains at least one polymer, it may also include a plasticizer. The plasticizer helps to keep the polymer flexible, and helps prevent it from forming a dry brittle film during usage of the coupling emulsion. Typical concentrations would be between 1-10%. Examples of suitable plasticizers include humectants such as glycols, glycerin, and polyethylene, such as polyethylene glycols that are liquid at room temperature.

### D. Waxes

Waxes as used herein refers to hydrophobic substances that are solids at room temperature. Waxes may be tolerated in the compositions of the invention, although oils are generally preferable. Waxes are generally acceptable in the emulsion compositions described herein as long as kept to low enough levels so as not to negatively affect rheology, and in particular so long as they do not cause unacceptable balling and pilling. Typically, a natural wax can be included in the composition at greater than 1%, or greater than 5%, or greater than 10% of the composition without significant adverse effect on the rheological properties of the emulsion.

Waxes can include natural, mineral and/or synthetic waxes. Natural waxes are those of animal origin, including without limitation beeswax, spermaceti, lanolin, and shellac wax, and those of vegetable origin, including without limitation carnauba, candelilla, bayberry, and sugarcane wax, and the like. Mineral waxes contemplated to be useful include, without limitation ozokerite, ceresin, montan, paraffin, microcrystalline, petroleum, and petrolatum waxes, synthetic waxes and polyolefin waxes, such as ethylene homopolymers, ethylene-propylene copolymers, and ethylene-hexene copolymers,

### E. Components to be Limited

Certain components are typically counter-indicated in the coupling emulsions described herein. These components are typically limited in the emulsion, and in certain embodiments the emulsion is free or substantially free of the component.

### Powders

Powders are generally limited in the coupling emulsions of the invention. Powders, particularly when provided in an emulsion with a volatile solvent, will produce a ‘dough-like’ material upon being subject to extended friction that accompanies the ultrasound method. Typically, the emulsion is substantially free of powder.

In specific embodiments in which a powder is included in the emulsion, a non-volatile solvent will also be included in the emulsion. When a polymer and a powder are included in the emulsion, they will collectively comprise no more than about 5% of the total emulsion, typically between about 0.01% to about 5% by weight of the emulsion, and more typically will comprise from about 0.01% to about 2.5%, or from about 0.025% to about 2%, and more typically still, from about 0.05% to about 1% by weight of the emulsion. In preferred embodiments, the combination of both a powder and a polymeric film former is avoided and where such a combination exists, the weight percentage of each is carefully limited, and preferably kept to very low levels. For example, in certain embodiments comprising both a powder and a polymeric film former, each of the powder and polymeric film former is preferably present in no more than about 0.1%. In more preferred embodiments, the composition is substantially free of any powder where a polymeric film former is present; and the composition is substantially free of any polymeric film former where a powder is present.

Powders to be limited in the coupling emulsion include hydrophobic organic powders, which include, but are not limited, to spherical or substantially spherical polymeric particles of polyethylene, polypropylene, polytetrafluoroethylene (PTFE), polyvinylchloride (PVC), polyvinyledenefluoride (PVDF), polyamide imide, polymethylmethacrylate (PMMA), polyetheretherketone (PEEK), polyethylene terephthalate polyester (PETP), polystyrene, polymethylsisesquiaxane, polyamide (Nylon) powder, methylsilsesquioxane resin microspheres; particles of polymethylsilsesquioxane; microspheres of polymethylmethacrylates; spherical particles of polymethylmethacryiate; particles of VinylDimethicone/Methicone Silsesquioxane Crosspolymer; spherical particles of crosslinked polydimethylsiloxanes; spherical particles of polyamide; polystyrene microspheres; aluminum starch octenylsuccinate; microspheres of polyethylene, spherical particles of PTFE; silicone resin, polymethylsilsesquioxane silicone polymer; Dimethicone/Divinyldirnethicone/Silsesquioxane Crosspolymer; platelet shaped powder made from N-lauroyl lysine; particulate silicon wax; particulate vinyl polymer; inorganic spherical particles include in particular alumina and silica, talc, sericite, mica, pigments such as titanium dioxide, iron oxide, boron nitride; and fatty acid derivatives of lysine. Powders that are especially to be limited or avoided in combination with polymeric film formers include materials that remain as powders in the composition, such as mica, talc, boron nitride and the like.

In one embodiment, a hydrophobic particle to be limited may be an oxide particle having its surface bound with non-polar radicals, such as for example alkyl groups, silicones, siloxanes, alkylsiloxanes, organosiloxanes, fluorinated siloxanes, perfluorosiloxanes, organosilanes, alkylsi lanes, fluorinated silanes, perfluorinated silanes and/or disilazanes and the like. Some particulate materials to be limited are hydrophobically modified metal oxides and metalloid oxides, including without limitation, titanium dioxide, iron oxides, tin dioxide, zinc oxide, zirconium dioxide, and combinations thereof.

Generally, components that should be limited in the coupling emulsion include particulates having a coefficient of dynamic (kinematic) friction, μk, greater than 0.5. Typically, if a powder is included in the emulsion, it will have a coefficient of dynamic friction less than 0.5, less than 0.45, less than 0.4, less than 0.35, less than 0.3, less than 0.25, less than 0.2, less than 0.15, or less than 0.1. One high dynamic friction particulate material to be limited is surface-modified aluminum oxide (Al₂O₃). Hydrophobically modified silica (SiO₂) powder, including fumed silica or pyrogenic silica (e.g., having a primary particle size range from about 7 nm to about 40 nm and an aggregate particle size between about 100 and about 400 nm) is also contemplated to be limited and in particular embodiments, the emulsion is substantially free of these.

### Polymeric Film Formers

The coupling emulsion can include one or more hydrophobic film formers, however the total amount of the film former is typically limited. In those embodiments in which polymeric film formers are incorporated in the emulsion, typically a non-volatile solvent is added in excess to reduce any pilling or balling from the film former. The emulsion typically includes less than five, such as four, three, two, or one film former. Typically the film former is in the emulsion at less than 2%, or less than 1% based on the entire weight of the emulsion. If powders are also present, then the film former is typically less than 0.5%, or less than 0.25%, or is entirely absent.

A film former is generally a hydrophobic material, and generally indicates a polymer which is capable, by itself or in the presence of at least one auxiliary film-forming agent, of forming a continuous film which adheres to a surface and functions as a binder for the particulate material. The term “hydrophobic” film-forming polymer will typically refer to a polymer with a solubility in water at 25° C. of less than about 1% by weight.

Film formers can be either natural or synthetic, polymeric or non polymeric, resins, binders, with low or high molar mass. Polymeric film formers can be either natural or synthetic, addition or condensation, homochain or heterochain, monodispersed or polydispersed, organic or inorganic, homopolymers or copolymers, linear or branched or crosslinked, charged or uncharged, thermoplastic or thermoset, elastomeric, crystalline or amorphous or both, isotactic or syndiotactic or atactic.

Polymeric film formers include polyolefins, polyvinyls, polyacrylates, polyurethanes, polyamides, polyesters, fluoropolymers, polyethers, polyacetates, polycarbonates, polyimides, rubbers, epoxies, formaldehyde resins, and homopolymers and copolymers of and of the foregoing. Typically, a polyurethane should be limited, and the emulsion is typically substantially free of polyurethanes, in particular when the emulsion contains a powder.

Additional film formers include copolymers comprising one or more blocks selected from styrene (S), alkylstyrene (AS), ethylene/butylene (EB), ethylene/propylene (EP), butadiene (B), isoprene (I), acrylate (A) and methacrylate (MA), or a combination thereof; certain polyalkylenes, and in particular C₂-C₂₀ alkene copolymers, such as polybutene; alkylcelluloses with a linear or branched, saturated or unsaturated C₁-C₈ alkyl radical, such as ethylcellulose and propylcellulose; copolymers of vinylpyrrolidone (VP) and in particular copolymers of vinylpyrrolidone and of C₂ to C₄₀ and better still C₃ to C₂₀ alkene, including the copolymers of vinyl pyrollidone with eicosene or dodecane monomers; polyanhydride resins; copolymers derived from maleic anhydride and C₃ to C₄₀ alkenes such as octadecene-1; and polymers and copolymers made from esters of vinylic acid monomers, methyl-, butyl-, isobutyl-, 2-ethylhexyl-, and lauryl-methacrylate, benzyl acrylates, phenyl acrylate, and the like. The alkyl group of these esters may be chosen, for example, from fluorinated and perfluorinated alkyl groups and amides of the acid monomers can be made such as (meth)acrylamides, for example. N-alkyl(meth)acrylamides, such as (C₁-C₂₀) alkyls, including without limitation, N-ethylacrylamide, N-t-octylacrylamide and N-undecylacrylamide.

Other film formers known in the art include acrylate copolymers, acrylates C₁₂₋₂₂ alkyl methacrylate copolymer, acrylate/octylacrylamide copolymers, actylate/VA copolymer, amodimethicone, AMP/acrylate copolymers, behenyl/isostearyl, butylated PVP, butyl ester of PVM/MA copolymers, calcium/sodium PVM/MA copolymers, dimethicone propylethylenediamine behenate, dimethicolnol ethylcellulose, ethylene/acrylic acid copolymer, ethylene/MA copolymer, ethylene/VA copolymer, fluoro C₂₋₈ alkyldimethicone, C₃₀₋₃₈ olefin/isopropyl maleate/MA copolymer, hydrogenated styrene/butadiene copolymer, hydroxyethyl ethylcellulose, isobutylene/MA copolymer, methyl methacrylate crosspolymer, methylacryloyl ethyl betaine/acrylates copolymer, octadecene/MA copolymer, octadecene/maleic anhydride copolymer, octylacrylamide/acrylate/butylaminoethyl methacrylate copolymer, oxidized polyethylene, perfluoropolymethylisopropyl ether, polyethylene, polymethyl methacrylate, polypropylene, PVM/MA decadiene crosspolymer, PVM/MA copolymer, PVP. PVP/decene copolymer, PVP/eicosene copolymer, PVP/hexadecene copolymer, PVP/MA copolymer, PVP/VA copolymer, sodium acrylate/vinyl alcohol copolymer, stearoxy dimethicone, stearoxytrimethylsilane, stearyl alcohol, stearylvinyl ether/MA copolymer, styrene/DVB copolymer, styrene/MA copolymer, tetramethyl tetraphenyl trisiloxane, tricontanyl PVP, trimethyl pentaphenyl trisiloxane, VA/crotonates copolymer, VA/crotonates/vinyl (proprionate copolymer, VA/butyl maleate/isobornyl acrylate copolymer, vinyl caprotactam/PVP/dimethylaminoethyl methacrylate copolymer, and vinyldimethicone.

Additional non-limiting representatives of hydrophobic film-forming polymers include polycondensate chosen from polyurethanes, polyurethane-acrylics, polyurethane-polyvinylpyrrolidones, polyester-polyurethanes, polyether-polyurethanes, polyureas and polyurea/polyurethanes. Additional non-limiting representatives of polycondensates may be chosen from polyesters, polyesteramides, fatty-chain polyesters, polyamides resins, epoxyester resins, arylsulphonamide-epoxy resins, and resins resulting from the condensation of formaldehyde with an arylsulphonamide.

Typically, silicone gums are limited in the emulsions. In certain embodiments, the emulsion does not include an acrylate/C₁₂₋₂₂ alkylmethacrylate copolymer. In certain embodiments, the film former is a silicone elastomer. Silicone elastomers are essentially tiny rubber particles swollen with solvent. Silicone elastomers swell in solvents such as dimethicone or cyclomethicone. Rubbing and/or heating causes the particles to lose solvent and coagulate, making elastomers prone to pilling/balling. Typically, the coupling emulsion is substantially free of silicone elastomers. To the extent that any silicone elastomer is included in the emulsion, the emulsion should include an excess of non-volatile solvent as compared to any volatile solvent included in the composition.

In some embodiments, the emulsions will be substantially free of alumina or hydrophobically modified alumina. Typically, the emulsion is substantially free of silica or hydrophobically-modified silica.

Polymeric viscosifying agents should generally be limited in the coupling emulsion. A viscosifying agent is typically one that provides the emulsion a viscosity of from about 1,000 mPas to about 1,000,000 mPas, typically from about 3,000 mPas to about 100,000 mPas. A viscosifying agent can include a carboxylic acid/carboxylate copolymer and a cellulose derivative polymer. Commercially available carboxylic acid/carboxylate copolymers include: CTFA name Acrylates/C10 30 Alkyl Acrylate Crosspolymer having tradenames Pemulen TR-1, Pemulen TR-2, Carbopol 1342, Carbopol 1382, and Carbopol ETD 2020, all available from B.F. Goodrich Company. Typically, the emulsion is substantially free of a polymeric viscosifying agent.

Cellulose derivative polymers should also generally be limited. These include methylcellulose, ethylcellulose, hydroxyethylcellulose, hydroxyethyl ethylcellulose, hydroxypropyl methyl cellulose, nitrocellulose, sodium cellulose sulfate, sodium carboxymethylcellulose, crystalline cellulose, cellulose powder, and mixtures thereof.

Additional water soluble polymers that should be limited include vinyl polymers such as cross linked acrylic acid polymers with the CTFA name Carbomer, pullulan, mannan, scleroglucans, polyvinylpyrrolidone, polyvinyl alcohol, guar gum, hydroxypropyl guar gum, xanthan gum, acacia gum, arabia gum, tragacanth, galactan, carob gum, karaya gum, locust bean gum, carrageenin, pectin, amylopectin, agar, quince seed (Cydonia oblonga Mill), starch (rice, corn, potato, wheat), algae colloids (algae extract), microbiological polymers such as dextran, succinoglucan, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid. Typically, the emulsion is substantially free of such agents.

Certain polyalkylene glycols should be limited. Typically, these have a molecular weight of more than about 1000 and include polyethylene oxides, polyoxyethylenes, polyethylene glycols, polypropylene oxides, polyoxypropylenes, polypropylene glycols, polypropylene glycols, mixed polyethylene-polypropylene glycols, or polyoxyethylene-polyoxypropylene copolymer polymers.

Polymeric film formers that are especially to be limited or avoided in combination with powders include polymeric gums, polymeric thickeners, cross-linked polymers, structuring polymers and, even more especially, the polymers referred to as “Film Formers” in the INCI Ingredient Dictionary and Handbook 11th Edition 2006, the disclosure of which is hereby incorporated by reference.

### F. Active Agent Delivery

Ultrasound can be used to deliver molecules to within the skin. When ultrasound is used in this context it is termed “sonophoresis”. Ultrasound applied to the skin has two main effects. First, cavitation results from the rapidly oscillating pressure field, causing bubble formation and collapse, which mechanically creates channels through the stratum corneum. The second effect is the direct heating of the material through which the sound waves are travelling, due to attenuation of the acoustic energy through reflection, absorption and dispersion. In skin, this occurs up to four times more than other tissues due to its heterogeneity. Heating is known to disrupt the lipid bilayer system in the stratum corneum also contributing to the enhanced permeability of the epidermis.

Ultrasound can be used to improve transdermal drug delivery. WO 99/34857 discloses transdermal drug delivery of various active agents using a power density of less than 20 W/cm², or less than 10 W/cm²; the frequency used being less than 2.5 MHz, less than 2 MHz, less than 1 MHz, or 20-100 kHz. The permeability of the skin is increased by disruption of the intercellular lipids through heating and/or mechanical stress, and through the increase in porosity. Temperature rises of 6° C. (1 MHz, 0.25 W/cm²) to 500C (20 kHz, 10-30 W/cm²) have been reported, but rises as little as 11° C. (1 MHz, 2 W/cm²) have been shown to cause skin damage. Continuous mode ultrasound at an intensity of 1 W/cm² raises the temperature of tissue at a depth of 3 cm to 40° C. in 10 minutes.

U.S. Pat. No. 4,767,402, describes transdermal drug delivery using ultrasound at a power density of 0-3 W/cm², 0.5-1.5 MHz, and recommends that as the power density is reduced, the frequency should also be reduced. A power density of 1-2 W/cm² at frequency 870 kHz is exemplified.

The present emulsion can also include a further skin active agent. The term “skin active agent” as used herein, means an active ingredient which provides a cosmetic and/or therapeutic effect. The skin active agents useful herein include skin lightening agents, anti-acne agents, emollients, non-steroidal anti-inflammatory agents, topical anesthetics, artificial tanning agents, antiseptics, anti-microbial and anti-fungal actives, skin soothing agents, sun screening agents, skin barrier repair agents, anti-wrinkle agents, anti-skirt atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, and mixtures thereof. In general, the present emulsion includes from about 0.001% to about 30%, typically from about 0.001% to about 10% of at least one skin active agent. Where appropriate, the amount of the active is, nonetheless, limited by the constraints discussed above on volatile solvents, powders, and polymers, and are included in any calculation of the aggregate amount of those ingredients.

The type and amount of skin active agents are selected so that the inclusion of a specific agent does not affect the stability of the emulsion. One benefit of the present coupling emulsion over the gels used as ultrasound coupling emulsions in the art is that, not only can water-soluble agents be used, water-insoluble agents or oil-soluble agents may also be included.

Treatment for cosmetic skin conditions, such as skin ageing and sun damage, typically delivers actives to at least the depth of the upper (papillary) dermis and therefore must employ a mechanism to overcome this effective physical and biochemical barrier, even when it has deteriorated with age.

The deterioration of human skin due to natural or ‘intrinsic’ ageing is characterized by a number of symptoms. Intrinsic aging is characterized by atrophy of skin with loss of elasticity and reduced metabolic activity. Typically, the stratum corneum remains relatively unchanged, but the epidermis thins overall, with a flattening of the dermal-epidermal junction resulting in increased fragility of the skin Dermal thickness and dermal vascularity are decreased; this is accompanied by a decrease in the number and the biosynthetic activity of dermal fibroblasts. Increasing age also has the effect of reducing the response of keratinocytes and fibroblasts to growth factors.

Visible deterioration in skin is characterized by: sagging skin, rough skin texture, dyspigmentation, dull complexion and a general loss of radiance. Wrinkling, or rhytide formation, is probably the symptom most commonly associated with skin ageing and is known to be caused by a change in the type and distribution of matrix proteins and proteoglycans. Similarly, functions of the skin that decline with age include: cell replacement, immune recognition, sensory perception, injury response, vascular responsiveness, vitamin D production, barrier function, thermoregulation, sebum production, chemical clearance, sweat production and mechanical protection. There may also be changes in pH (from 4.5 to 5).

The skin is also subjected to environmental ageing processes. For example, factors such as diet, pollution and smoking are known to affect the rate of skin ageing. However one factor stands out as the most potent ‘gerontogen’: sunlight. It has been suggested that approximately 80% of facial ageing is due to sun exposure. Collagen, elastin and other intra- and extracellular proteins of the skin are affected resulting in solar elastosis, the build-up of localized elastic tissue in fibrous bundles throughout the dermis. The UV component of sunlight has also been linked to the reduction in cellular population of the epidermis (keratinocytes) and dermis (fibroblasts). It has been suggested that this is due to the increase in programmed cell death or apoptosis. The epidermis and the dermis are known to become increasingly acellular with age, which supports this hypothesis. Despite the epidermis influencing the dry and rough appearance of the skin, it is the dermis that dictates the degree of surface smoothness. Reduction and/or a redistribution of matrix proteins and high water-binding proteoglycans largely govern the appearance of wrinkles and general surface smoothness. Similarly, scarring of the skin is due to abnormal protein content, conformation and distribution via the formation of granulation tissue following trauma, again primarily a dermal rather than an epidermal problem.

Typical symptoms of photoageing include coarseness, wrinkling, irregular pigmentation, telangiectasia, scaliness and a variety of benign, premalignant and malignant neoplasms. Photoageing is predominant in fair-skinned Caucasians who have a history of sun-exposure and occurs most severely on the face, neck and extensor surfaces of the upper extremities. Elastosis, recognized as the pebbly goose flesh seen on the neck and upper chest, is due to nodular aggregations of altered elastin fibers in the dermis. A proliferation of increasingly thickened and tangled elastin fibers has been observed in the papillary and reticular dermis of sun-exposed skin. Even in mildly sun-damaged skin, a 5-20 fold increase in elastin fiber diameter has been found, with slight changes in the fibrillar structure and an alteration of the normal architecture, giving a disrupted and “moth-eaten” appearance.

At the molecular and ultrastructural level, there are changes in elasticity and other changes in matrix proteins. As regards elasticity, there is a reduction in the extracellular protein fibrillin which is a major component of microfibril bundles that connect the dermal-epidermal junction to the papillary dermis. These bundles, often called oxytalan fibers, essentially provide an elastic connection between the epidermis and dermis. Previously considered to be synthesized only by fibroblasts, the fibers present at the dermal-epidermal junction have been shown to be synthesized by keratinocytes. The concentration of fibrillin photoaged skin has been found to be decreased and has proved to be a useful biomarker for photoageing as it is known to be connected with wrinkle formation. Fibrillin concentration is also reduced in skin that has been subjected to tensile stress and exhibits stretch marks (striae distensae).

Heat Shock Proteins (HSPs), also known as stress proteins, are thought to act as molecular chaperones by assisting with protein synthesis, transport, folding and degradation. They are a group of proteins that are present in all cells, in all life forms. They are induced when a cell undergoes environmental stress, heat, cold, or oxygen deprivation. HSPs are also present in cells under normal conditions and have been linked to modulation of contraction and relaxation responses in vascular smooth muscle; they play an important role in protein folding and function, even in the absence of stress.

Temperature rises of 3-5° C. above baseline in muscle have been shown to cause the induction of HSPs. Induction of HSPs by 30 mins of pulsed ultrasound applied at normal body temperature has been demonstrated in the rat embryo, showing that the heat shock response is not specific to heat but can occur in response to mechanical stress. Similarly, chick embryos exposed to ultrasound, without any significant thermal contribution, have shown heightened synthesis of HSP72 suggesting that the mechanical stimulus can induce a stress response. It was also concluded that to produce a full biological effect, stress must be constant for approximately 10 s or more over any time interval during exposure'. It is possible that cumulative effects can stimulate HSP production as has been found when mild heat shock was repeated over 3 days causing significantly elevated muscle HSP levels.

Analgesics such as aspirin, ibuprofen and paracetamol are known to protect against cataract. This action has been attributed to the inhibition of sugar-induced cross-linking in small HSPs such as α-crystallin Enzymes that protect against cataract are prone to glycation-induced inactivation, but aspirin has been shown to protect against this.

Similarly, acetyl-L-carnitine has been recognized as a potential chaperone-protecting agent due to its abilities to acetylate potential glycation sites of small HSPs and correspondingly protect them from glycation-mediated protein damage.

Small heat shock proteins (sHSPs) and Clusterin are molecular chaperones that share many functional similarities despite their lack of significant sequence similarity. Small heat shock proteins are ubiquitous intracellular proteins whereas clusterin is generally found extracellularly. Both chaperones prevent the amorphous aggregation and precipitation of target proteins under stress conditions such as elevated temperature, reduction and oxidation, Transcription of both HSPs and clusterin are mediated by the transcription factor HSF-1. However, clusterin has been shown to be much more efficient than certain sHSPs, such as α-crystallin, in preventing the precipitation from solution of stressed target proteins.

Coupling emulsions of the invention are useful in the treatment of cosmetic skin conditions, in particular acting to improve the appearance of ageing skin, especially by ameliorating the effects of sun damage.

Other particularly useful additional ingredients are sunscreens, Sunscreens are typically those with a broad range of UVB and UNA protection, such as octocrylene, avobenzone (Parsol 1789), octyl methoxycinnamate, homosalate benzophenone, camphor derivatives, zinc oxide, and titanium dioxide. Inorganic powder uv blockers are not preferred for the reasons previous set forth, Emulsions can have about 0.01 wt % to about 50 wt % sunscreens based on the total weight of the emulsion or about 0.1 wt % to about 40 wt % or about 1 wt % to about 30 wt % sunscreens. When sunscreen is present, typical would be emulsions with about 15-50% sunscreen, or emulsions which provide an SPF of 15, 25, 30, 40, 50, or greater than 50. In certain embodiments, the additional ingredient is not a sunscreen.

Other particularly useful additional ingredients are exfoliating agents, such as alphahydroxyacids, betahydroxyacids, oxaacids, oxadiacids, and their derivatives such as esters, anhydrides and salts thereof. A typical exfoliating agent is glycolic acid. Typical emulsions have about 0.1 wt % to about 80 wt % exfoliating agents based on the total weight of the emulsion. More common emulsions have about 1 wt % to about 40 wt % exfoliating agents. Most typically, emulsions have about 1 wt % to about 15 wt % exfoliating agents.

Other particularly useful additional ingredients are additional anti-inflammatories. The anti-inflammatories may be of synthetic, natural or semi-synthetic origin. The anti-inflammatories may be steroidal or non-steroidal. Useful examples include, but are not limited to, mangostin, eysenhardtia polistachya (Palo Azul) wood extract, rosemary extract, camphor, salicylates, hydrocortisone, aspirin, indomethacin, mefenamic acid and derivatives thereof.

It has been shown that topical anti-inflammatory agents can reduce chronic photodamage, which produces many of the signs of aging such as wrinkles and fine lines, as well as hyperpigmentation. In one study, topical hydrocortisone, ibuprofen, and naproxen were tested against photodamage. Calcineurin inhibitors block the inflammation process by reducing phosphorylation of NFAT, leading to reduced T cell stimulation, Topical calcineurin inhibitors have been used to treat certain severe skin inflammatory reactions, such as atopic dermatitis (see e.g. Fume, et al. (2006) Dermatol. Ther. 19:118-26). Calcineurin inhibitors have been used on sensitive skin, such as the face and eyelids, to replace the need for corticosteroids in such diseases or to otherwise reduce the potential side effects associated with corticosteroids. Specifically, both pimecrolimus (Elidel) cream and tacrolimus (Protopic) ointment are available.

Typical emulsions have about 0.01 wt % to about 25 wt anti-inflammatories based on the total weight of the emulsion. More commonly, emulsions have about 0.1 wt % to about 15 wt % anti-inflammatories. Most typically, emulsions have about 0.5 wt. % to about 10 wt % anti-inflammatories.

Agents for incorporation into coupling emulsions include one or more of a histidine containing dipeptide, alanyl-L-histidine (L-carnosine) or a peptidomimetic thereof, N-acetylcysteine, aminoguanidine, d-penicillamine, acetylsalicyclic acid (aspirin), paracetamol, indomethacin and ibuprofen and/or a functional homolog; derivative or prodrug thereof. Histidine-containing natural dipeptides, such as L-carnosine (β-alanyl-L-histidine, or “carnosine”), or related compounds including imidazole, histidine, N-acetyl-L-carnosine (NAC), anserine, β-aianylhistamine (carcinine), N-acetyl-β-alanylhistamine (N-acetyl carcinine), h-prolyl histamine, and/or n-acetyl-L-carnosine are known to be effective against different oxygen-derived free radicals, and also lipoperoxyl radicals. Carnosine, present at high concentrations in skeletal muscle tissue, can delay senescence and provoke cellular rejuvenation in cultured human fibroblasts. The mechanism by which such a simple molecule induces these effects is not known despite carnosine's well documented anti-oxidant and oxygen free-radical scavenging activities. In addition to the prophylactic actions of carnosine, it may also directly participate in the inactivation/disposal of aged proteins possibly by direct reaction with the carbonyl groups on proteins. The possible fates of these camosinylated proteins include the formation of inert lipofuscin, proteolysis via the proteasome system and exocytosis following interaction with receptors

An emulsion according to the invention can include one or more anti-oxidant(s). The antioxidant can be selected from: arginine, ascorbic acid, a prodrug or derivative of ascorbic acid, ascorbyl palmitate, magnesium ascorbyl phosphate, trisodium ascorbyl phosphate, anserine, carnosine, opidine, homocarnosine and/or acetylanserine. Generally, the anti-oxidants are usually present at from about 0.5 to 5%, typically from about 1 to 3% w/w of the emulsion.

Coupling emulsions may contain one or more substances capable of inducing expression of a molecular chaperone, particularly useful are substances capable of inducing expression of a heat shock protein, clusterin and/or alpha crystallin. The one or more substance capable of inducing expression of a molecular chaperone can be acetyl salicylic acid, salicylic acid, zinc ions, a zinc salt, zinc sulfate, and/or zinc-L-carnosine. Usually, a zinc containing agent is present at from about 0.1 to 1%, or from about 0.25 to 0.75%, or around 0.5% w/w of the emulsion. When acetyl salicylic acid or salicylic acid is present in the emulsion a suitable concentration is from about 0.5 to 2.5%, or from about 1 to 1.5% w/w of the emulsion.

The coupling emulsion can also include one or more anti-apoptotic substance, typically selected from the group comprising nicotinoamide, L-carnitine, acetyl-L-carnitine, N-acetyl-cysteine and/or L-carnosine. An anti-apoptotic substance is usually present at a concentration of from about 0.5 to 5%, or 1 to 3% of the emulsion.

The coupling emulsion can also include one or more substance capable of inducing expression of a molecular chaperone and a dermatologically acceptable excipient.

The coupling emulsion can also include one or more ingredient selected from one or more vitamins, one or more small peptide(s), and/or one or more amino acid(s) Or a derivative or prodrug thereof. Vitamins that may be incorporated into emulsions of the invention include vitamin B compounds such as thiamine (vitamin B1), e.g. as thiamine pyrophosphate, such as benfotiamine; pyridoxamine (vitamin B6), vitamin A and/or F, or a derivative or prodrug thereof.

The emulsion may include one or more small peptides) suitably as a dipeptide, tripeptide and/or tetrapeptide, and/or one or more amino acid(s), e.g. proline, lysine, histidine, alanine, or a derivative or prodrug thereof.

The emulsion may further include one or more polysaccharide, which may be one or more proteoglycan, such as a glycosaminoglycan. The one or more glycosaminoglycan employed can be a low and/or high molecular weight hyaluronan, chondriotin sulphate, dermatan sulphate and/or one or more derivative(s) thereof. Some glycosaminoglycans, especially Hyaluronic Acid or Hyaluronan (“HA”), have been shown to be decreasingly present in ageing skin. These substances are known to influence migration, growth and differentiation of connective tissue cells in some instances. HA is a long-chained polysaccharide that is a major constituent surrounding cells in most animal tissues. HA has been used for decades in cosmetics, viscosurgery and viscosupplementation without immunological reactions or any other side-effects.

In one embodiment, an emulsion will include a low and high molecular weight HA and/or one or more derivative(s) thereof. Low molecular weight HA characteristically has a molecular weight of less than 1×10⁶ Da, whereas a high molecular weight hyaluronan generally has molecular weight of greater than 1×10⁶ Da. The HA molecule can be derivatized via modification of the acetamido, the reducing end group but most commonly the hydroxy and carboxylate groups. The glycosidic bond is also readily hydrolyzed to create shorter chains or oligosaccharides. HA-drug adducts have been synthesized for controlled delivery applications and HA-protein adducts as biomaterials and cell substrates. Low-molecular weight HA (˜300 kDa) is available from Sigma, Poole, Dorset (isolated from bovine vitreous humor). High molecular weight HA is available from ConvaTec, Flintshire, UK (isolated from human umbilical cord). Other HAs include NIF-NaHA marketed under the name of Healon™ for medical and Hylartil™ for veterinary use; Hylan A (elastoviscous fluid) and Hylan B (viscoelastic gel) developed by Biomatrix Inc.

Skin lightening agents are generally active ingredients that improve hyper-pigmentation as compared to pre-treatment. Useful skin lightening agents include ascorbic acid compounds, vitamin B₃ compounds, azelaic acid, butyl hydroxyanisole, gallic acid and its derivatives, glycyrrhizinic acid, hydroquinone, kojic acid, arbutin, mulberry extract, and mixtures thereof. Ascorbic acid compounds include, ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives include, for example, esters of ascorbic acid, and ester salts of ascorbic acid. Ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include: magnesium ascorbyl phosphate available from Showa Denko, 2-o-D-glucopyranosyl-L-ascorbic acid available from Hayashibara and sodium L-ascorbyl phosphate with tradename STAY C available from Roche. Vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide.

Other skin active agents include panthenol, tocopheryl nicotinate, benzoyl peroxide, 3-hydroxy benzoic acid, flavonoids flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinal, retinyl esters (e.g., retinyl propionate), phytic acid, N-acetyl-L-cysteine, lipoic acid, tocopherol and its esters (e.g., tocopheryl acetate), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4-trichlorocarbanitide, octopirox, lidocaine hydrochloride, clotrimazole, miconazole, ketoconazole, neomycin sulfate, theophylline, TDPA, its salts and/or esters, and mixtures thereof. Any of these may be beneficially included in the coupling emulsion described for use in the present system.

The invention described and claimed herein is not to be limited in scope by the specific embodiments disclosed since these embodiments are intended as illustrations of aspects of the invention. Any equivalent embodiments are intended to be within the scope of this invention. Indeed, various modifications in addition to those described will become apparent to those skilled in the art from the foregoing description. Such modifications are also intended to fall within the scope of the claims. All publications cited herein are incorporated by reference in their entirety.

### EXAMPLES

### Example 1

### Measurement of Tan(Delta) of Pilling and Non-Pilling Compositions

Samples of ‘pilling’ and ‘non-pining’ compositions were measured to determine their rheological characteristics. Each sample was measured twice fresh (i.e. from the jar) at 25° C. at a gap of 500 μm. Then a film of each sample was artificially stressed (heated for 30 minutes at 50° C.) and measured at 37° C. at a gap of 10 μm.

For measurement of fresh compositions, samples of compositions were held between parallel plates (40 mm in diameter, at a gap of 500 μm) and oscillated at a frequency of 1 s⁻¹ as the applied stress was gradually increased. The increasing stress results in increasing strain or deformation as the theological properties were measured G′, G″, tan(delta). The elastic modulus ((F) is directly correlated to the stiffness of the sample while the viscous modulus (G″) accounts for the liquid-like component. The tan(delta) or tangent of the phase angle is equal to the ratio of G″ to G′. Tan(delta)<1 is indicative of a solid-like sample while tan(delta)>1 is indicative of a liquid-like sample. The “yield stress” of the composition is defined as the oscillatory stress when tan(delta)=1, that is, when G″/G′=1.

For measurement of stressed compositions, a 40 mil (1.02 mm in thickness) wet film of the sample was placed on the bottom plate of the instrument. The sample was then heated at 50° C. for 30 minutes to drive off the volatiles and “age” the composition. The top plate (20 mm in diameter and serrated to prevent the sample from slipping) was then lowered to a 10-μm gap before the measurement was conducted. The 10-μm gap size was chosen based on other data that suggests the dried film is thicker than 10-μm (ensuring sample contact).

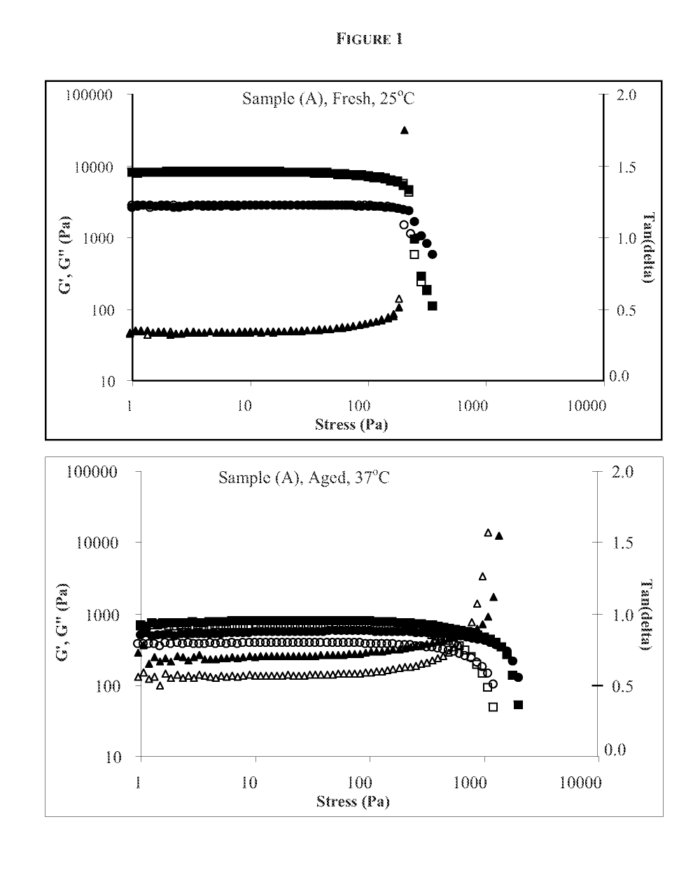

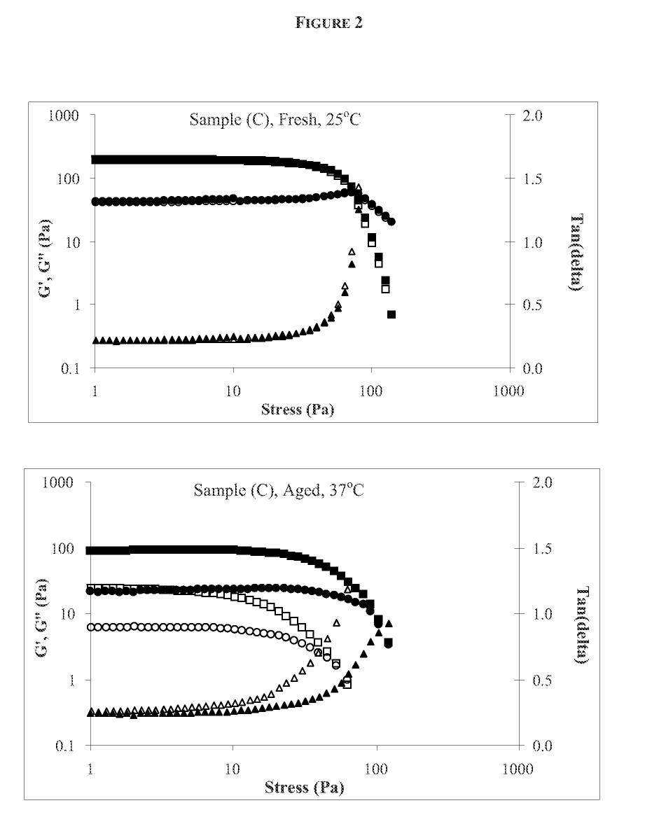

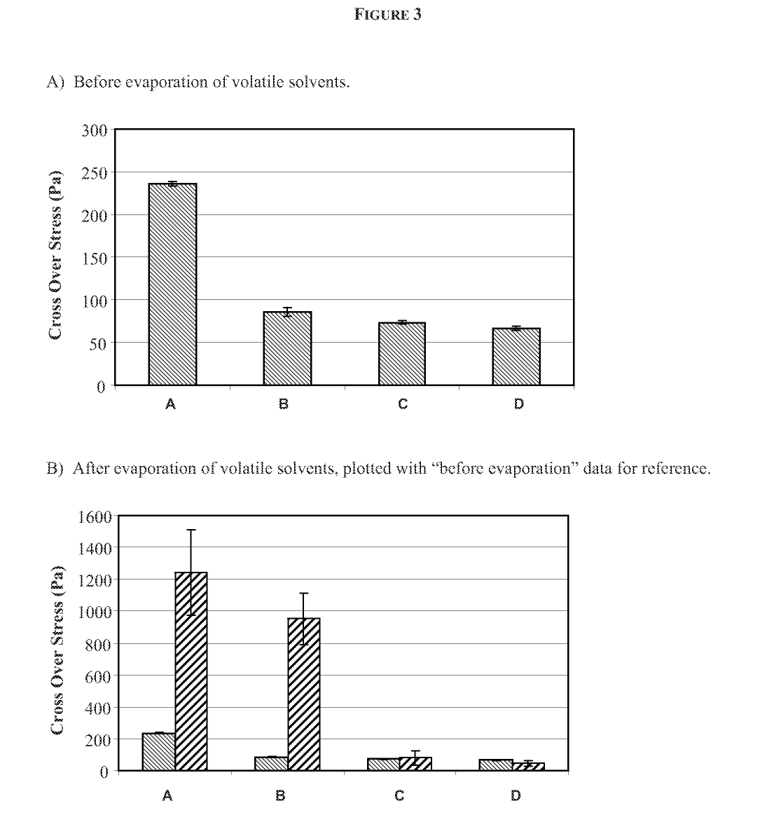

FIGS. 1 and 2 are representative plots of stress sweeps of new versus aged compositions for pilling and non-pilling compositions, respectively. FIG. 3 is a graph showing the combined data from experiments on four compositions, two of which will pill (A) and (B) and two of which will not pill (C) and (D) when used in the system described herein, i.e. when used as a coupling emulsion in conjunction with an ultrasound probe causing friction on skin for between 2 and 10 minutes. In the fresh state, the yield stresses of the 4 samples are similar (FIG. 3a). Once aged and at a smaller gap, the pilling samples show a dramatic increase in “yield stress” (FIG. 3b). Table 1 provides the data used to generate FIG. 3.

TABLE 1
Stress at tan(delta) = 1

| | Fresh, | | | | | | |
|---|---|---|---|---|---|---|---|
| | 25° C., | Fresh, | Avg. | Aged, | Aged, | Avg. | % |
| | t₁ | 25° C., t₂ | Stress | 37° C., t₁ | 37° C., t₂ | Stress | Change |
| A | 234 | 238 | 236 | 1435 | 1056 | 1246 | 428 |
| B | 82 | 89 | 86 | 1066 | 837 | 952 | 1013 |
| C | 75 | 72 | 74 | 112 | 46 | 79 | 7 |
| D | 69 | 65 | 67 | 59 | 35 | 47 | −30 |

Compositions

Pilling Samples

A. Anew Ultimate Night Creme (Commercially available product.)

B. Anew Retroactive Day Creme (Commercially available product.)

Non-Pilling Samples

| | Samples | |
|---|---|---|
| | C | D |
| water | 67.43 | 66.23 |
| chelators | 0.05 | 0.05 |
| polyethylene glycol 400 (humectant) | 4.00 | 4.00 |
| glycerin (humectant) | 3.00 | 5.00 |
| acrylates/C10-30 alkyl acrylate crosspolymer | 0.82 | 0.82 |
| (polymeric emulsifier/thickener/film former) | | |
| (Trade name Pemulen TR-1, TR-2, from Noveon) | | |
| pH adjustor | 0.80 | 0.80 |
| emollient oils | 12.00 | 12.00 |
| waxes | 0.25 | 0.25 |
| emulsifiers | 1.55 | 1.55 |
| dimethicone/cetearyl dimethicone crosspolymer/ | 1.00 | — |
| peg/ppg-20/23 dimethicone (blend of silicone | | |
| elastomer, emulsifier, diluent) | | |
| (Trade name Y-17483 from Momentive) | | |
| dimethicone fluid-volatile (Trade namd SF96-5 from | 3.00 | 3.00 |
| Momentive) | | |
| dimethicone fluid-nonvolatile (Trade name SF96-350 | 0.10 | 0.10 |
| from Momentive) | | |
| active ingredients | 5.80 | 6.00 |
| fragrance | 0.20 | 0.20 |
| Total | 100.00 | 100.00 |

1-34. (canceled)

35. A skin treatment system comprising:
(i) a hand-held device having a surface configured to be brought into contact with the skin for transmitting energy to the skin;
(ii) a coupling emulsion for providing a lubricious surface between said device surface and said skin; said coupling emulsion comprising an aqueous phase, an oil phase, and an emulsifier for stabilizing said emulsion;
wherein, when the emulsion is stressed, said coupling emulsion has a rheology: characterized by a yield stress value (a) of less than 300 Pa, and/or (b) that does not increase by more than 10%.

36. The skin treatment system according to claim 1, wherein the rheology is Characterized after evaporation of the volatile solvents from the coupling emulsion.

37. The skin treatment system according to claim 36, wherein, when the emulsion is stressed, the yield stress value does not increase by more than 10% and is less than 300 Pa, and wherein said emulsion is a water-in-oil emulsion or an oil-in-water emulsion.

38. The system according to claim 37, wherein said emulsion is a water-in-silicone emulsion or a silicone-in-water emulsion, and wherein the oil phase of said emulsion comprises a non-volatile silicone fluid having a viscosity of greater than about 5 centistokes at 37° C.

39. The system according to claim 36, wherein the collective weight of all particulate materials in said emulsion is less than 1% by weight of said emulsion and the collective weight of all polymeric film formers is less than 2% by weight of said emulsion, and wherein said emulsion comprises one or more active ingredients for providing a therapeutic or cosmetic benefit to the skin.

40. The system of claim 36, wherein said device includes an element that transmits heat energy into the skin.

41. The system according to claim 36, wherein said device is an ultrasound device which transmits ultrasonic energy into said skin.

42. A kit comprising:
(i) a coupling emulsion for providing a lubricious surface between the skin and a surface of a hand-held device configured to be brought into contact with the skin; said coupling emulsion comprising an aqueous phase, an oil phase, and an emulsifier for stabilizing said emulsion; wherein, when the emulsion is stressed, said coupling emulsion has a rheology after evaporation of volatile solvents: characterized by a yield stress value (a) of less than 300 Pa and/or (b) that does not increase by more than 10%; and
(ii) written instructions for using said coupling emulsion to provide a lubricious surface between said skin and said surface of said hand-held device configured to be brought into contact with the skin for transmitting energy to said skin surface.

43. The kit according to claim 42, wherein, when the emulsion is stressed, the yield stress value does not increase by more than 10% and is less than 300 Pa, and wherein said emulsion is a water-in-oil emulsion or an oil-in-water emulsion.

44. The kit according to claim 43, wherein said emulsion is a water-in-silicone emulsion or silicone-in-water emulsion, and wherein the oil phase of said water-in-silicone emulsion or said silicone-in-water emulsion comprises a non volatile silicone fluid having a viscosity of greater than about 5 centistokes at 37° C.

45. The kit according to claim 12, wherein the collective weight of all particulate materials in said emulsion is less than 1% by weight of said emulsion and the collective weight of all polymeric film formers is less than 2% by weight of said emulsion, and wherein said emulsion comprises one or more active ingredients for providing a therapeutic or cosmetic benefit to the skin.

46. The kit according to claim 42, further including a device for transmitting heat energy to the skin.

47. The kit according to claim 42, wherein said device is an ultrasound device which transmits ultrasonic energy into said skin.

48. A method for treating the skin comprising:
(i) applying to the skin a coupling emulsion for providing a lubricious surface between the skin and a surface of a hand-held device configured to be brought into contact with the skin; said coupling emulsion comprising an aqueous phase, an oil phase, and an emulsifier for stabilizing said emulsion; wherein, when the emulsion is stressed, said coupling emulsion has a rheology after evaporation of volatile solvents: characterized by (a) a yield stress value of less than 300 Pa, and/or (b) that does not increase by more than 10%; and
(ii) contacting said skin with said surface of a hand-held device to transmit enemy to said skin.

49. The method according to claim 48, wherein said yield stress value does not increase by more than 10% when the emulsion is stressed and is less than 300 Pa when the emulsion is stressed, and wherein said emulsion is a water-in-oil emulsion or an oil-in-water emulsion.

50. The method according to claim 19, wherein said emulsion is a water-in-silicone emulsion or a silicone-in-water emulsion and the oil phase comprises a non-volatile silicone fluid having a viscosity of greater than about 5 centistokes at 37° C.

51. The method according to claim 48, wherein the collective weight of all particulate materials in said emulsion is less than 1% by weight of said emulsion and the collective weight of all polymeric film formers is less than 2% by weight of said emulsion and wherein said emulsion comprises one or more active ingredients for providing a therapeutic or cosmetic benefit to the skin.

52. The method according to claim 48, wherein said treatment is repeated daily for a period sufficient to reduce the average wrinkle depth in the skin area and/or to reduce discoloration in the skin area.

53. The system of claim 48, wherein said device includes an element that transmits heat energy into the skin.

54. The method according to claim 48, wherein said device is an ultrasound device which transmits ultrasonic energy into said skin.

55. The method according to claim 48, wherein said coupling emulsion remains substantially free of balling and pilling during use.
